(19) United States
(12) Patent Application Publication
(10) Pub. No.: US 2009/0030277 A1
(43) Pub. Date: Jan. 29, 2009

(54) Endoscope Insertion Portion and Endoscope System

(76) Inventors: Ryuhei Fujimoto, Tokyo (JP); Yasuhito Kura, Tokyo (JP); Ryuichi Toyama, Tokyo (JP); Katsutaka Adachi, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/887,990

(22) PCT Filed: Mar. 31, 2006
(86) PCT No.: PCT/JP2006/306904

§ 371 Date: Oct. 5, 2007

(30) Foreign Application Priority Data
Apr. 5, 2005 (JP) 2005-109096
Jan. 13, 2006 (JP) 2006-006782

(51) Int. Cl. A61B 1/00

(52) U.S. Cl. 600/114

(57) ABSTRACT

To realize an endoscope insertion portion and endoscope system capable of providing a significant propulsive function by reducing a friction between a propulsive force generation section and an insertion portion, an endoscope insertion portion comprising: an introductory tube as an insertion portion; a spiral tube as a propulsive force generation section mounted on an outer peripheral surface of the introductory tube and rotating around the longitudinal axis of the introductory tube; and a plurality of ring members provided on an outer peripheral surface of an elastic cover tube at predetermined intervals between an inner peripheral surface of a spiral tube and the elastic cover tube 21, as a friction reduction section positioned between the spiral tube and the outer peripheral surface of the introductory tube (elastic cover tube) and reducing a contact resistance between an outer peripheral surface of the introductory tube (elastic cover tube) and the spiral tube.

### BACKGROUND OF THE INVENTION

1. Field of the Invention

The present invention relates to an endoscope insertion portion to be introduced into a body cavity and an endoscope system.

2. Description of the Related Art

In recent years, endoscopes have been widely employed in the medical-application field. Such an endoscope can observe an affected area or the like in a body cavity by inserting a slender endoscope insertion portion into the body cavity and, as needed, perform treatments and procedures by inserting a treatment instrument into a forceps channel. The endoscope has a bending portion being bendable on the distal end side of the endoscope insertion portion. The bending portion of the endoscope is bendable in the vertical or horizontal direction by operating a bending operation knob.

When the endoscope is inserted into a complicated body cavity, for example, a tube cavity drawing a 360° loop like a large intestine, the bending portion is bent by operating the bending operation knob and, at the same time, an twisting operation is performed, so that the endoscope insertion portion is inserted toward a portion to be observed. However, such an operation of an endoscope requires a skill to a degree that the insertion portion can be smoothly inserted up to a deep portion of the complicated large intestine in a short time.

An inexperienced operator may lose track of an insertion direction while inserting the insertion portion to a deep region, which may cause the operator to confront insertion difficulty or largely deviate its running state in an intestine from a target route. Accordingly, there have conventionally been a variety of proposals for enhancing the insertability of the endoscope insertion portion.

For example, Japanese Patent Laid-Open Publication No. Hei 10-113396, hereinafter referred to as Patent Document 1, has disclosed a propulsion system for medical instrument capable of guiding a medical instrument easily and with little invasion up to the deep region of a biological duct. The propulsion system is formed with a slanting rib on a rotation member in the rotational axis direction as a propulsive force generation section. Accordingly, the propulsion system in Patent Document 1 described above rotates the rotation member, so that a rotational force of the rotation member is converted into a propulsion force by the rib and the medical instrument connected to the propulsion system is moved toward the depth direction by the propulsion force. This permits the propulsion system in Patent Document 1 described above to insert the medical instrument into a body cavity with a slight invasion and without giving a physical burden to a patient.

The propulsion system for medical instrument described in Patent Document 1 has a hollow cylindrical body formed with the rotation member at the distal end and the hollow cylindrical body is slidably provided on the inner peripheral surface of an endoscope insertion portion.

However, the propulsion system for a medical instrument described in Patent Document 1 generates friction between the hollow cylindrical body formed with the rib and an inner peripheral surface of the endoscope insertion portion. With the propulsion system described in Patent Document 1, the friction may inhibit the cylindrical body from rotating, so that the propulsion system may not exhibit propulsion function satisfactorily.

In view of the above-described problems, it is an object of the present invention to provide an endoscope insertion portion and an endoscope system capable of attaining a significant propulsion function by reducing friction between a propulsive force generation section and an insertion portion.

### SUMMARY OF THE INVENTION

In order to achieve the above object, a first endoscope insertion portion according to the present invention comprises: an insertion portion capable of being inserted into a subject; a propulsive force generation section mounted on an outer peripheral surface of the insertion portion and rotating around a longitudinal axis of the insertion portion; and a friction reduction section provided between the propulsive force generation section and the outer peripheral surface of the insertion portion and reducing a contact resistance between the outer peripheral surface and the propulsive force generation section.

A second endoscope insertion portion according to the present invention comprises: an insertion portion capable of being inserted into a subject; a propulsive force generation section fitted onto the insertion portion rotatably around the longitudinal axis of the insertion portion and self-propelling the insertion portion inserted into a body cavity by rotation; and a friction reduction section for reducing a contact resistance between an inner peripheral surface of the propulsive force generation section and an outer peripheral surface of the insertion portion by setting a distance between the inner peripheral surface of the propulsive force generation section and the outer peripheral surface of the insertion portion not being constant.

An endoscope system according to the present invention comprises: a slender and flexible endoscope insertion portion; a flexible insertion portion guide member mounted on the outer periphery side of the endoscope insertion portion and formed with a rotatable spiral-shaped portion on an outer peripheral surface; a rotation device for rotating the spiral-shaped portion of the insertion portion guide member around the longitudinal axis; and a friction reduction section for reducing a contact resistance between the spiral-shaped portion rotated by the rotation device and an outer periphery of the insertion portion guide member.

### BRIEF DESCRIPTION OF THE DRAWINGS

FIG. 1 is a view showing an overall configuration of an endoscope system in a first embodiment;

FIG. 2 is an external view showing the vicinity of a distal end portion of an introductory tube in FIG. 1;

FIG. 3 is a descriptive view showing an introductory tube and an endoscope in FIG. 1;

FIG. 4 is a sectional view taken on line A-A in FIG. 3;

FIG. 5 is a descriptive view showing a configuration of a rotation mechanism portion;

FIG. 6 is a descriptive view of a substantial part showing the vicinity of a distal end portion of an introductory tube in FIG. 2;

FIG. 7 is a descriptive view showing such a state that the introductory tube inserted with an insertion portion is inserted from an anus;

FIG. 8 is a descriptive view showing such a state that a distal end portion of the introductory tube inserted with an insertion portion is inserted up to the vicinity of a caecum portion;

FIG. 9 is a descriptive view of a substantial part showing the vicinity of a distal end portion of an introductory tube of a first variant in FIG. 6;

FIG. 10 is an enlarged view of a substantial part in FIG. 9;

FIG. 11 is a descriptive view of a substantial part showing the vicinity of a distal end portion of an introductory tube of a second variant in FIG. 6;

FIG. 12 is a descriptive view of a substantial part showing the vicinity of a distal end portion of an introductory tube of a third variant in FIG. 6;

FIG. 13 is a descriptive view of a substantial part showing the vicinity of a distal end portion of an introductory tube configuring an endoscope system in a second embodiment;

FIG. 14 is an enlarged view of a substantial part showing a first variant in FIG. 13;

FIG. 15 is an enlarged view of a substantial part showing a second variant in FIG. 13;

FIG. 16 is a descriptive view of a substantial part showing the vicinity of a distal end portion of an introductory tube configuring an endoscope system in a third embodiment;

FIG. 17 is a descriptive view of a substantial part showing the vicinity of a distal end portion of an introductory tube of a first variant in FIG. 16;

FIG. 18 is a descriptive view of a substantial part showing the vicinity of a distal end portion of an introductory tube of a second variant in FIG. 16;

FIG. 19 is a sectional view of the substantial part in FIG. 18;

FIG. 20 is a descriptive view of a substantial part showing the vicinity of a distal end portion of an introductory tube configuring an endoscope system in a fourth embodiment;

FIG. 21 is a descriptive view of a substantial part showing the vicinity of a distal end portion of an introductory tube of a first variant in FIG. 20;

FIG. 22 is a descriptive view of a substantial part showing the vicinity of the distal end portion of the introductory tube of the second variant in FIG. 20;

FIG. 23 is a descriptive view of a substantial part showing the vicinity of a distal end portion of an introductory tube configuring an endoscope system in a fifth embodiment;

FIG. 24 is a descriptive view of a substantial part showing the vicinity of a distal end portion of an introductory tube in FIG. 23;

FIG. 25 is a descriptive view of a substantial part showing the vicinity of a distal end portion of an introductory tube configuring an endoscope system in a sixth embodiment;

FIG. 26 is a descriptive view of a substantial part showing the vicinity of a distal end portion of an introductory tube configuring an endoscope system in a seventh embodiment;

FIG. 27 is an external perspective view showing a broad metal sheet as a raw material of a plate-shaped metal member;

FIG. 28 is an outline view showing such a state that the metal sheet in FIG. 27 is cut to plate-shaped metal members with small width using a cutter;

FIG. 29 is a schematic view showing such a state that burring is generated on a plate-shaped metal member in a cutting operation in FIG. 28;

FIG. 30 is an external perspective view showing a plate-shaped metal member formed in cutting operations in FIGS. 28 and 29;

FIG. 31 is a descriptive view in plastically deforming plate-shaped metal members in FIG. 30 using a metal mold;

FIG. 32 is an outlined perspective view showing part of a spiral tube formed by engaging a plastically deformed plate-shaped metal members with another plate-shaped metal member and winding them around a core member (not shown); and

FIG. 33 is an outlined sectional view showing an operation of the spiral tube formed in FIG. 32.

### BEST MODE FOR CARRYING OUT THE INVENTION

Referring now to drawings, embodiments according to the present invention will be described below.

### First Embodiment

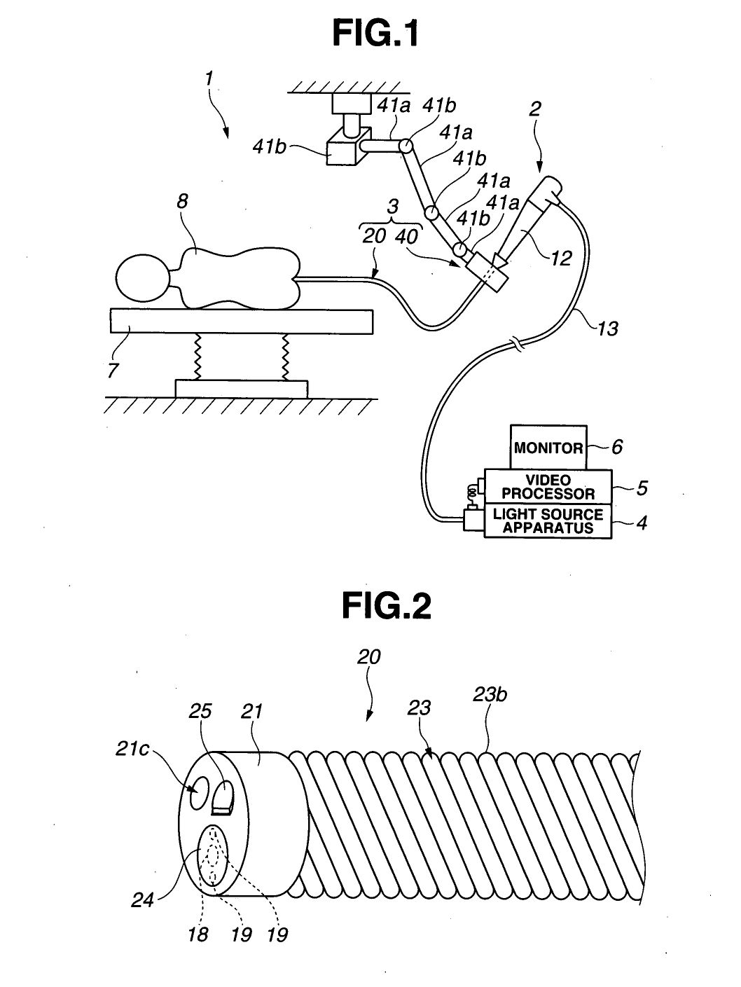

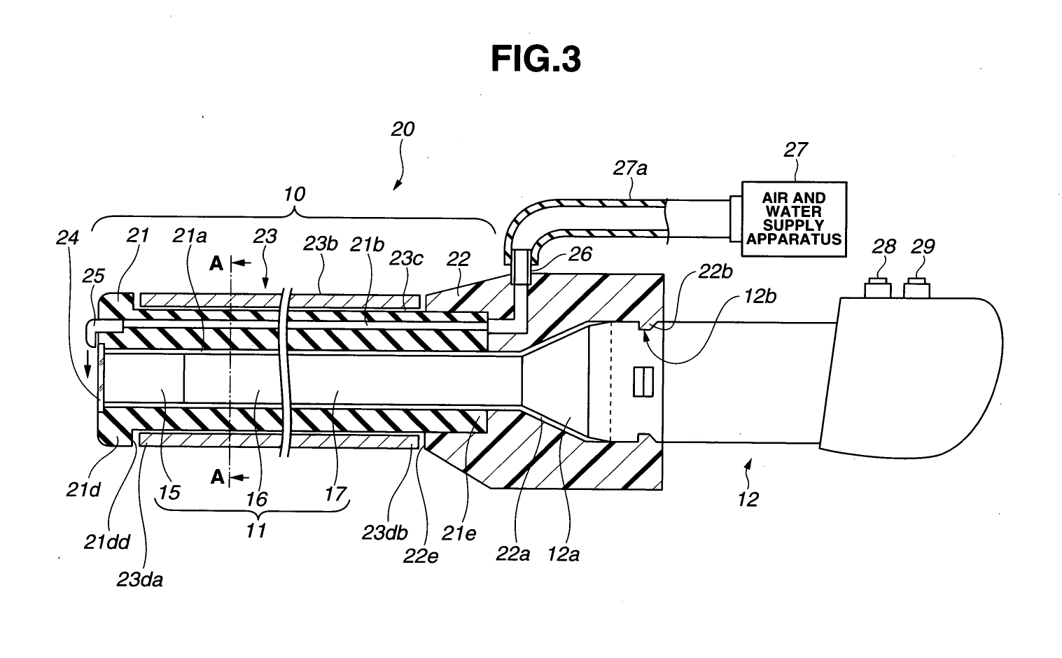

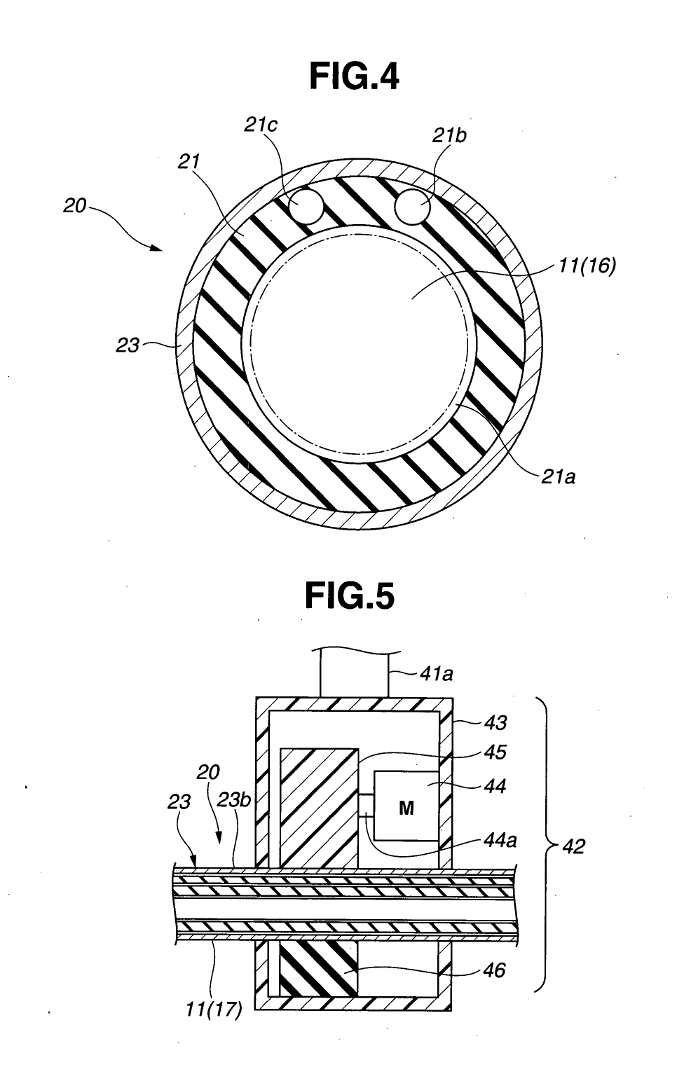

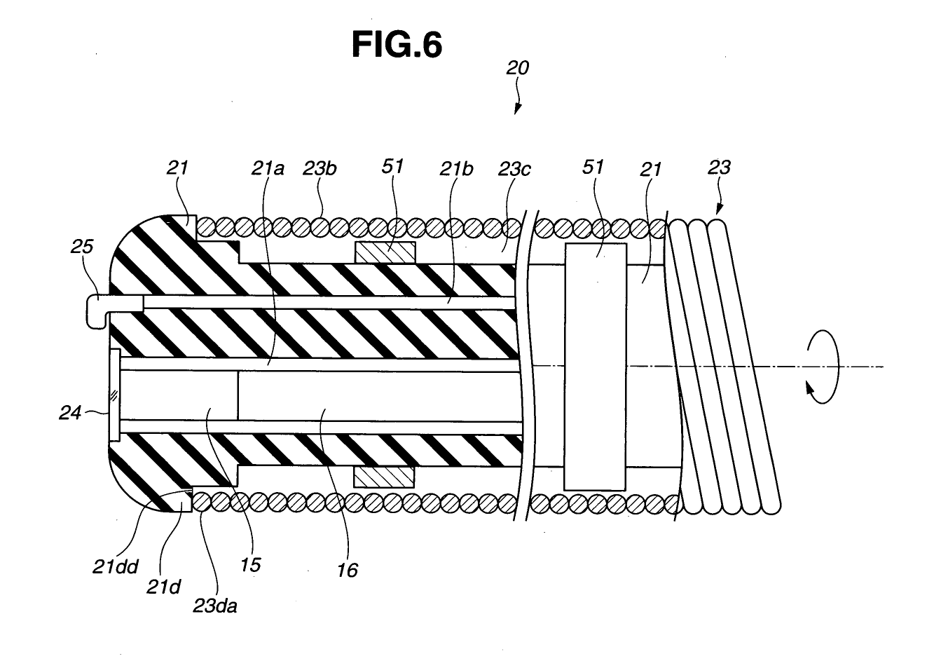

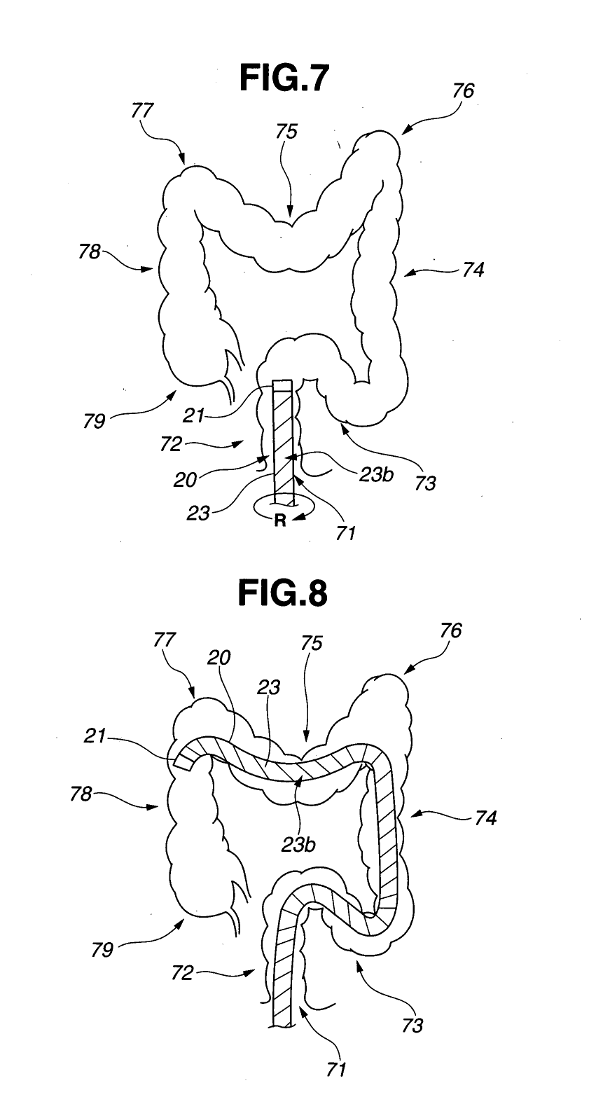

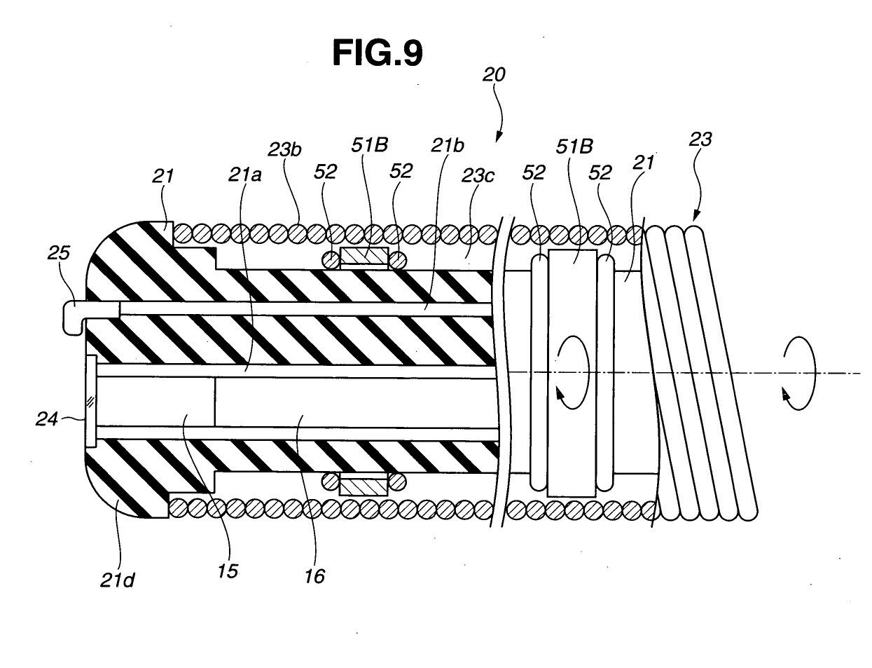

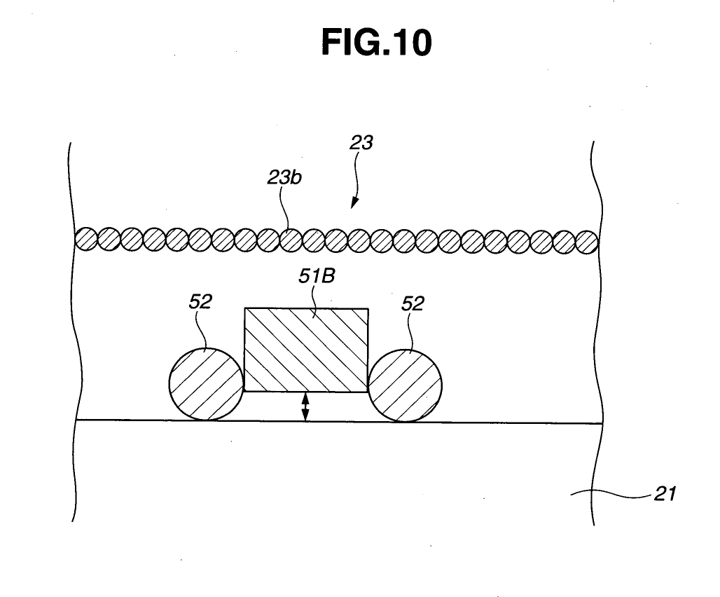

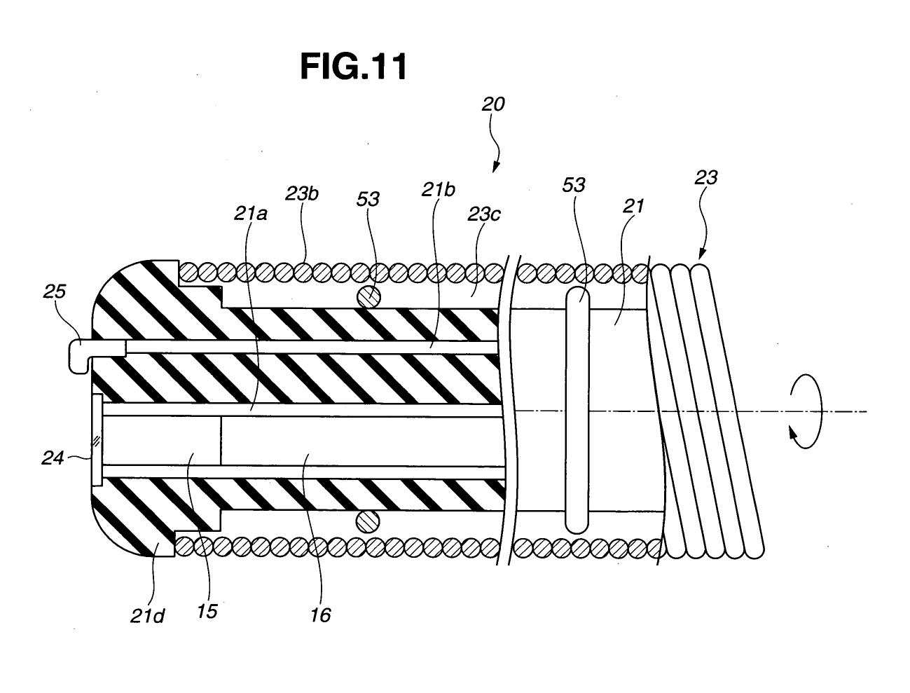

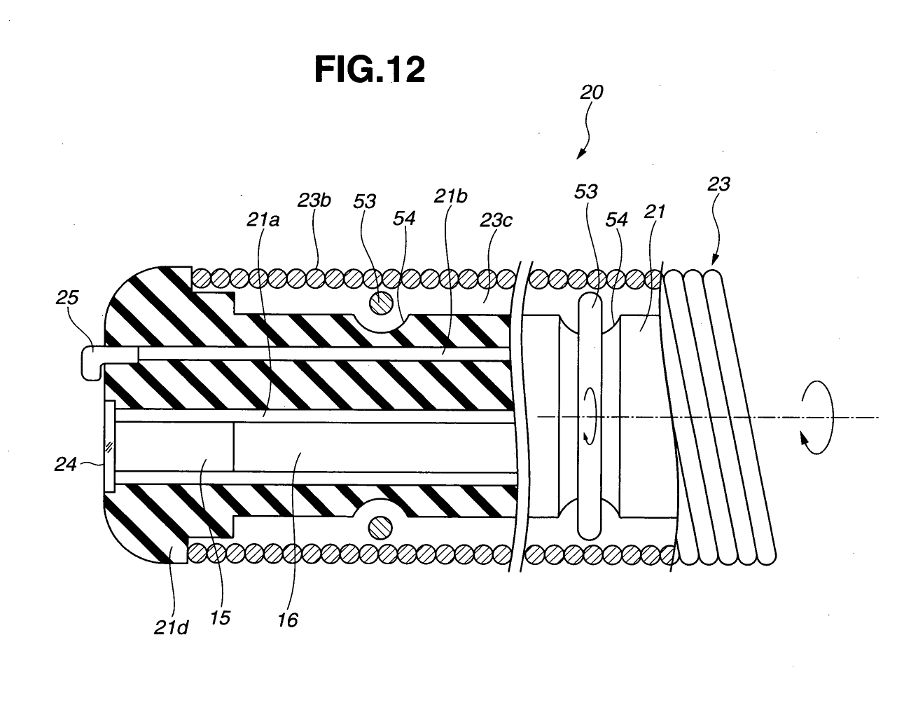

FIGS. 1 to 12 relate to a first embodiment according to the present invention. FIG. 1 is a view showing an overall configuration of an endoscope system in a first embodiment, FIG. 2 is an external view showing the vicinity of a distal end portion of an introductory tube in FIG. 1, FIG. 3 is a descriptive view showing the introductory tube and an endoscope in FIG. 1, FIG. 4 is a sectional view taken on line A-A in FIG. 3, FIG. 5 is a descriptive view showing a configuration of a rotation mechanism portion, FIG. 6 is a descriptive view of a substantial part showing the vicinity of a distal end portion of an introductory tube in FIG. 2, FIG. 7 is a descriptive view showing such a state that the introductory tube inserted with an insertion portion is inserted from an anus, FIG. 8 is a descriptive view showing such a state that a distal end portion of the introductory tube inserted with an insertion portion is inserted up to the vicinity of a caecum portion, FIG. 9 is a descriptive view of a substantial part showing the vicinity of a distal end portion of an introductory tube of a first variant in FIG. 6, FIG. 10 is an enlarged view of a substantial part, FIG. 11 is a descriptive view of a substantial part showing the vicinity of a distal end portion of an introductory tube of a second variant in FIG. 6, and FIG. 12 is a descriptive view of a substantial part showing the vicinity of a distal end portion of an introductory tube of a third variant in FIG. 6.

As shown in FIGS. 1 to 3, an endoscope system 1 in the present embodiment is composed of an endoscope 2 and an endoscope insertion assisting tool 3. The endoscope 2 is connected to a light source apparatus 4, a video processor 5 and a monitor 6 and the like. The endoscope 2 is supplied with illumination light from the light source apparatus 4, and the illumination light illuminates a subject. The endoscope 2 takes in an image of the illuminated subject from an objective lens system (not shown) and outputs an image pickup signal photoelectrically converted by an image pickup device to the video processor 5. The video processor 5 processes the image pickup signal from the image pickup device, produces a video signal, and outputs the video signal to the monitor 6 to display an endoscope image.

The endoscope 2 has a slender and flexible endoscope insertion portion 11, an operation portion 12 provided on the proximal end side of the endoscope insertion portion 11 and a universal cord 13 extending from the side portion of the operation portion 12. The endoscope insertion portion 11 is constituted by consecutively installing a rigid distal end portion 15, a bendable bending portion 16 and a long flexible tube portion 17 in order from the distal end side.

The operation portion 12 is provided with a bending operation knob (not shown) for bending the bending portion 16. The endoscope 2 is configured so that the bending portion 16 is freely bendable by operating the bending operation knob. An introductory tube 20 inserted with the endoscope 2 and serving as a guide tube described later is configured so as to bend, following a bending operation of the bending portion 16 of the endoscope 2.

The endoscope insertion assisting tool 3 comprises an introductory tube 20 as an insertion portion for guiding the endoscope insertion portion 11 which is inserted in the depth direction of a body cavity and a rotation device 40 for rotating a spiral tube 23 described later of the introductory tube 20.

The rotation device 40 comprises an arm 41 one end of which is attached onto a ceiling of an inspection room and a rotation mechanism portion 42 mounted on the other end of the arm portion 41. The arm portion 41 is composed of a plurality of arm members 41a, for example, having different lengths and a plurality of joint portions 41b pivotally connecting the arm members 41a adjacent to each other.

This permits the rotation device 40 to move the rotation mechanism portion 42 to a desired position with a slight effort. A detailed configuration of the rotation mechanism portion 42 will be described later.

As shown in FIGS. 2 to 4, the introductory tube 20 comprises an insertion portion cover 10 constituted of an observation window member 24 and an elastic cover tube 21, a proximal-end-side component 22 consecutively installed on the insertion cover 10, and a spiral tube 23 of a rotating cylindrical body disposed on the outer periphery side of the insertion portion cover 10 and serving as a propulsive force generation section for generating a propulsive force. In other words, the introductory tube 20 as an insertion portion is formed with the spiral tube 23 of a rotating cylindrical body disposed onto the outer-periphery surface side of the introductory tube 20 and serving as a propulsive force generation section rotating around a longitudinal axis of the introductory tube 20.

The elastic cover tube 21 is formed in an elongated tubular shape with low frictional resistance, for example, fluoroethylene resin such as PTFE (tetrafluoroethylene resin). The elastic cover tube 21 has a through hole 21a in which the endoscope insertion portion 11 is inserted and which axially penetrates the tube 21.

Moreover, the elastic cover tube 21 has a through hole 21b as an air and water supply channel, which axially penetrates the tube 21. Furthermore, the elastic cover tube 21 is formed with a through hole 21c axially penetrating as a treatment instrument insertion channel or suction channel as shown in FIG. 4.

At the front face of the elastic cover tube 21 on the distal end side, an observation window member 24 is disposed by adhesion or the like integrally with the elastic cover tube 21 at an opening portion of the through hole 21a on the distal end side. The proximal end side of the through hole 21a is communicated with a through hole 22a described later, which is formed at the proximal-end-side component 22.

The observation window member 24 is formed of a transparent resin material with optical properties, such as polycarbonate. When the endoscope insertion portion 11 is inserted into the through hole 21a, the front face of a distal end portion 15 constituting a part of the endoscope insertion portion 11 is made to abut against the inner-side surface of the observation window member 24. The observation window member 24 watertightly blocks the opening in the front face of the elastic cover tube 21 and serves as an observation window for the endoscope 2.

One end side of the through hole 21b is communicated with an air and water supply nozzle 25 disposed near the distal end portion of the elastic cover tube 21. The opening of the air and water supply nozzle 25 faces the observation window member 24.

On the other end of the through hole 21b, there is formed a mouth ring 26 so as to protrude to the outer periphery of the proximal-end-side component 22. One end of an air and water supply tube 27a is coupled to the mouth ring 26.

On the other end side of the air and water supply tube 27a, the air and water supply apparatus 27 is connected. The air and water supply apparatus 27 can be driven and controlled by depressing an air and water push-button switch 28.

The air and water supply apparatus 27 can be driven by depressing the air and water supply push-button switch 28, supply fluid such as air or liquid to the through hole 21b and jet fluid from an opening of the air and water supply nozzle 25 toward a surface of the observation window member 24 as indicated by an arrow.

If filth or the like adheres to the surface of the observation window member 24, this permits the introductory tube 20 to jet water from the opening of the air and water supply nozzle 25 to wash away the adhering filth. By supplying air from the opening of the air and water supply nozzle 25, the introductory tube 20 can remove water droplets adhering to a surface of the observation window member 24.

The through hole 21c is communicated with a channel opening portion formed at a predetermined site of the proximal-end-side component 22. In using the through hole 21c as a treatment instrument insertion channel, a treatment instrument such as a biopsy needle or a biopsy forceps is inserted into the channel opening portion.

The treatment instrument is inserted into the through hole 21c and is protruded from the distal end opening of the elastic cover tube 21 to perform a predetermined treatment. In using the through hole 21c as a suction channel, one end of a duct line connecting member is disposed at the channel opening, while the other end of the duct line connecting member is connected, for example, to a suction duct line (not shown) extending from a suction device (not shown).

The suction device can perform drive control by depressing a suction push-button switch 29. This permits the introductory tube 20 to suck body fluid in a body cavity from the distal-end opening of the cover tube 21 by a suction action of the suction device.

Accordingly, the endoscope 2 is formed with only an observation window 18 constituting an observation optical system and an illumination window 19 constituting an illumination optical system provided on a distal end surface of the endoscope insertion portion 11, thus minimizing the diameter of the endoscope insertion portion 11.

The spiral tube 23 is formed by winding metal strand of predetermined diameter in a spiral manner so as to have predetermined flexibility. The metal strand is constituted of, for example, stainless steal. On an outer surface of the spiral tube 23, a spiral-shaped portion 23b is constituted of a surface of metal strand.

The spiral tube 23 is constituted by forming a clearance 23c between an inner peripheral surface of the spiral-shaped portion 23b and an outer peripheral surface of the elastic cover tube 21 and covering an outer peripheral surface of the elastic cover tube 21 and is rotatably disposed in a peripheral direction (around its axis) relative to an outer peripheral surface of the elastic cover tube 21.

The spiral tube 23 is configured so as to rotate in a peripheral direction (around its axis) with the rotation mechanism portion 42 of the rotation device 40 as described later.

The spiral tube 23 is not limited to a one-row configuration. For example, a spiral tube wound in a plurality of rows such as two-row and four-row may be used. The spiral tube 23 can be adjusted in propulsive force and traveling speed by changing a close contact between metal strands or variously setting spiral angles in forming the spiral tube by winding the metal strand in a spiral manner.

At a distal end portion of an outer peripheral surface of the elastic cover tube 21, there is formed a protrusion portion 21d for preventing the spiral tube 23 from falling off. The spiral tube 23 is configured so that its front end portion 23da is made to abut against and latched by a rear face portion 21dd of the protrusion 21d, thus regulating forward movement of the spiral tube 23.

While its rear end portion 23bd is made to abut against and latched by a front face portion 22e of the proximal-end-side component 22, thus regulating backward movement of the spiral tube 23.

Accordingly, the spiral tube 23 is configured so that the front end portion 23da and the rear end portion 23db are latched by the rear face portion 21dd of the protrusion portion 21d on the front end side and by the front face portion 22e of proximal-end-side component 22 on the rear end side, respectively. This always maintains a state covering the outer surface side of the elastic cover tube 21.

On the other hand, the proximal-end-side component 22 of the insertion portion cover 10 is a tubular member having a larger diameter than that of the elastic cover tube 21 and is formed of resin material having high slidability, for example, boriacetar. Inside the proximal-end-side component 22, the through hole 22a is drilled so that a part (a part of a bend preventing portion 12a) of the operation portion 12 of the endoscope 2 on the distal end side may be inserted.

On an inner peripheral surface of the through hole 22a on the rear end side, there are protruded the plurality of latching protrusion portions 22b formed so as to protrude inward. The plurality of latching protrusion portions 22b are configured so as to be fitted into a peripheral groove 12b formed in the bend preventing portion 12a of the operation portion 12 of the endoscope 2.

Accordingly, the introductory tube 20 securely retain the endoscope 2 by fitting the plurality of latching protrusion portions 22b into the peripheral groove 12b when the endoscope insertion portion 11 is inserted into the elastic cover tube 21 and a part of the operation portion 12 on the distal end side is placed inside the proximal-end-side component 22.

A front face portion 22e of the proximal-end-side component 22 is fitted onto a part of the proximal-end portion 21e of the elastic cover tube 21. This permits the elastic cover tube 21 to be formed integrally with the proximal-end-side component 22.

As shown in FIG. 5, the rotation mechanism portion 42 has a rotation section body 43 as a housing, a motor 44, a torque transmission member 45 and an introductory tube retainer 46. The motor 44 produces a driving force for rotating the spiral tube 23 around the longitudinal axis (hereinafter referred to as “around an axis”). The motor 44 is secured onto, for example, a side wall of a rotation section body 43.

On a motor shaft 44a of the motor 44, the torque transmission member 45 is integrally fixed. The torque transmission member 45 is formed of flexible resin material. The introductory tube retainer 46 is disposed so as to face the torque transmission member 45 fixed on the motor shaft 44a.

The guide tube retainer 46 is secured onto, for example, a bottom portion of a rotation section body 43. A flat portion facing the torque transmission member 45 of the guide tube retainer 46 is formed with a semicircular recessed portion (not shown) substantially coinciding with an external shape of the spiral tube 23 or the proximal-end-side component 22. In the rotation mechanism portion 42, the spiral tube 23 constituting the introductory tube 20 is disposed in a sandwiched manner between the torque transmission member 45 and a recessed portion in the introductory tube retainer 46.

Accordingly, in the introductory tube 20, when the motor 44 is driven with the spiral tube disposed between the torque transmission member 45 and the introductory tube retainer 46, the torque transmission member 45 fixed onto the motor shaft 44a rotates and the rotational driving force is transmitted to the spiral tube 23 through the torque transmission member 45.

The spiral tube 23 to which torque is transmitted is rotated around an axis of the elastic cover tube 21 in a clearance 23c formed between an inner peripheral surface of the spiral-shaped portion 23b and the elastic cover tube 21.

In the introductory tube 20, the rotation of the spiral tube 23 produces such a propulsive force that an external thread (male thread) moves to an internal thread (female thread) at a contact portion between the spiral-shaped portion 23b and an intestine wall when inserted into a body cavity. This permits the spiral tube 23 to attempt to move in an axial direction of the introductory tube 20 while rotating.

At this time, the position of one end (front end portion 23da) of the spiral tube 23 is regulated at an abutment position against the protrusion portion 21d on the elastic cover tube 21, while the other end (rear end portion 23db) thereof is regulated at an abutment position against the front face portion 22e of the proximal-end-side component 22. This integrates the spiral tube 23 with the elastic cover tube 21. Accordingly, the elastic cover tube 21 is configured so as to move in the same direction as the spiral tube 23 as the spiral tube 23 moves.

At this time, in the introductory tube 20, the elastic cover tube 21 and the endoscope 2 are integrated with each other by fitting the latching protrusion portion 22b into the peripheral groove 12b under a condition as shown in FIG. 3, that is, a condition where the endoscope insertion portion 11 is inserted into the elastic cover tube 21 and the latching protrusion portion 22b is fitted into the peripheral groove 12b.

Accordingly, the endoscope 2 is configured so as to move in the same direction as a movement direction of the introductory tube 20 composed of the spiral 23 and the elastic cover 21 and advance toward a deep region of a body-cavity inner tube line. This permits the spiral tube 23 as a propulsive force generation section to self-propel the introductory tube 20 as an insertion portion inserted into the body cavity by rotation.

The introductory tube 20 is inserted into the winding body-cavity inner tube line while being bent. Accordingly, in the introductory tube 20, the spiral tube 23 under a rotating condition may be twisted within the winding body-cavity inner tube line, so that an inner peripheral surface of the spiral tube 23 and the elastic cover tube 21 as the outer periphery of an insertion-portion guide member might be brought into contact.

In this case, the spiral tube 23 generates a friction at a contact portion between an inner peripheral surface of the spiral tube 23 and an outer peripheral surface of the elastic cover tube 21, which will impair rotation around the axis. Furthermore, the spiral tube 23 will have the difficulty in transmitting torque by the rotation device 40 from a contact portion between an inner peripheral surface of the spiral tube 23 and an outer peripheral surface of the elastic cover tube 21 to the distal end portion.

Accordingly, the introductory tube 20 may be incapable of achieving a predetermined propulsive force at a contact portion between the spiral-shaped portion 23b and an intestine wall, resulting in an unsatisfactory propulsion function.

The present embodiment is configured to comprise a friction reduction section for reducing a contact resistance of the outer peripheral surface with the spiral tube 23, provided between an inner peripheral surface of the spiral tube 23 and an outer peripheral surface (non-rotation portion) of the elastic cover tube 21.

As shown in FIG. 6, between an inner peripheral surface of the spiral tube 23 and an outer peripheral surface (non-rotation portion) of the elastic cover tube 21, a plurality of ring members 51 to be irregularities as a friction reduction section are bonded, for example, over the range from the distal end side to the proximal end side of an outer peripheral surface of the elastic cover tube 21 at predetermined intervals using a fixing agent such as adhesives.

The friction reduction section does not necessarily need to be provided within the range of the distal end side to the proximal end side. The ring members 51 are respectively constituted of a material which has biological compatibility and high slidability, such as fluororesin such as PTFE, polyethylene or stainless steel. Furthermore, it is more effective that the ring members 51 are constituted of a material softer than that of metal strand of the spiral tube 23.

Accordingly, the introductory tube 20, having the plurality of ring members 51 over the range from the distal end side to the proximal end side on the outer peripheral surface of the elastic cover tube 21, prevents an inner peripheral surface of the spiral tube 23 and an outer peripheral surface of the elastic cover tube 21 from contacting each other over the full length by bringing the inner peripheral surface of the spiral tube 23 into contact with the plurality of ring members 51 even if the spiral tube 23 under a rotating condition gets twisted in a winding body-cavity inner tube line. At this time, the introductory tube 20 is configured so that a distance between the inner peripheral surface of the spiral tube 23 and the outer peripheral surface of the elastic cover tube 21 is not constant over the range from the distal end side to the proximal end side.

Thus, a contact area between the inner peripheral surface of the spiral tube 23 and the elastic cover tube 21 decreases, which enables the introductory tube 20 to reduce a contact resistance occurring therebetween.

The ring members 51 may be constituted by coating with fluororesin such as PTFE for high slidability. On the other hand, the outer peripheral surface of the elastic cover tube 21 is constituted by providing a material with high slidability, for example, fluororesin such as PTFE, polyethylene or gore tube.

The operation of an endoscope system 1 configured in the above way will be described below.

First, medical staff (hereinafter referred to as “staff”) prepares the endoscope 2 and the introductory tube 20 constituting the endoscope insertion assisting tool 3. The staff moves the arm portion 41 of the rotation device 40 constituting the endoscope insertion assisting tool 3 and locates the rotation mechanism portion 42 at a desirable position.

Next, the staff locates a desirable portion of the spiral tube 23 constituting the introductory tube 20 between the guide tube retainer 46 and the torque transmission member 45 constituting the rotation mechanism portion 42. This allows the proximal end side of the introductory tube 20 to be retained by the rotation mechanism portion 42. At this time, the staff locates the proximal end portion side of the introductory tube 20, for example, on a bed 7.

Next, the staff inserts the endoscope insertion portion 11 into the introductory tube 20 from an opening in the proximal-end-side member 22 constituting the introductory 20. Hence, the endoscope 2 completes preparatory work for, for example, performing insertion into, for example, a large intestine, with the introductory tube 20 covering the endoscope insertion portion 11.

The staff prepares the light source apparatus 4, the video processor 5 and the monitor 6 as peripheral apparatuses together with the endoscope 2, the introductory tube 20 and the rotation device 40.

Next, a step of inserting the endoscope 2 covered by the introductory tube 20 into a large intestine will be described below. First, an operator (not shown) holds the distal end of the introductory tube 20 and inserts the distal end of the introductory tube 20 into the large intestine of a patient 8 lying on a bed 7 from the patient's anus.

In the introductory tube 20 the distal end of which is inserted into the anus of the patient 8, the spiral-shaped portion 23b formed on an outer surface of the spiral tube 23, comes into contact with the patient's intestine wall. At this time, the spiral-shaped portion 23b has such a relationship with the intestine wall as seen in between an external thread (male thread) and an internal thread (female thread). On a screen of the monitor 6, an endoscope image picked up through the observation window 18 by an image pickup device of the endoscope 2 is displayed.

The operator rotates the motor 44 of the rotation mechanism portion 42 by a predetermined operation under a such a condition that the spiral-shaped portion 23b comes into contact with the intestine wall. The rotation mechanism 42, when the motor 44 performs rotational driving, rotates the torque transmission member 45 through the motor shaft 44a.

A rotational driving force of the torque transmission member 45 is transmitted to the spiral tube 23 disposed between the torque transmission member 45 and the guide tube retainer 46. Accordingly, as indicated by an arrow R in FIG. 7, the spiral tube 23 starts rotation around the axis.

At this time, the spiral-shaped portion 23b of the spiral tube 23 under a rotating condition has such a relationship at the contact portion with the intestine wall that an external thread moves with respect to an internal thread, that is, a propulsive force for advancing the spiral tube 23 is generated. As described above, the one end position (front end portion 23da) of the spiral tube 23 is regulated at an abutment position against the protrusion portion 21d of the elastic cover tube 21, while the position of the other end (rear end portion 23db) is regulated at an abutment position against the front face portion 22e of the proximal-end-side component 22, so that the spiral tube 23 is integrated with the elastic cover tube 21.

Accordingly, the spiral tube 23 is made to abut against the rear face portion 21dd of the protrusion portion 21d of the elastic cover tube 21 and advances while pressing it, without dropping off the elastic cover tube 21. This permits the introductory tube 20 composed of the spiral tube 23 and an elastic cover tube 21 to be advanced toward the deep region within the large intestine by the generated propulsive force.

At this time, in the proximal-end-side component 22 of the introductory tube 20, fitting the latching protrusion portion 22b onto the peripheral groove 12b integrates the endoscope 2. Accordingly, as the introductory tube 20 moves, the endoscope 2 as well moves in the same direction and inserted toward the deep region in the body cavity of a subject.

Under this condition, when the operator performs manual maneuvering to push forward the introductory tube 20, the introductory tube 20 with the endoscope insertion portion 11 inserted is to be introduced toward the deep portion in the body cavity with slight effort. That is to say, the introductory tube 20 inserted from an anus 71 with the endoscope insertion portion 11 inserted is advanced toward an S-shaped colon portion 73 from a rectum 72 by the propulsive force, operator's manual maneuvering, bending maneuvering and the like.

As described above, the introductory tube 20 is provided with the plurality of ring members 51 to be irregularities as a friction reduction section between the inner peripheral surface of the spiral-shaped portion 23b and the elastic cover tube 21 of the outer periphery of an insertion-portion guide member over the range from the distal end side to the proximal end side on an outer peripheral surface of the elastic cover tube 21 at predetermined intervals.

Accordingly, the introductory tube 20 prevents the inner peripheral surface of the spiral tube 23 and the outer peripheral surface of the elastic cover tube 21 from contacting each other over the full length by bringing the inner peripheral surface of the spiral tube 23 into contact with the plurality of ring members 51 even if the spiral tube 23 under a rotating condition gets twisted in a winding body-cavity inner tube line. Furthermore, at this time, the introductory tube 20 is set so that a distance between the inner peripheral surface of the spiral tube 23 and an outer peripheral surface of the elastic cover tube 21 is not constant over the range from the distal end side to the proximal end side.

Thus, a contact area between the inner peripheral surface of the spiral tube 23 and the elastic cover tube 21 decreases, which enables the introductory tube 20 to reduce a contact resistance occurring therebetween. Thus, the introductory tube 20 exerts a significant propulsion function when being inserted into the body cavity, so that the endoscope insertion portion 11 can be easily inserted into the body cavity.

On the introductory tube 20, filth or the like may adhere to the observation window member 24. In this case, the operator depresses the air and water supply push-button switch 28 twice.

The introductory tube 20 jets, for example, water as indicated by an arrow from the opening in the air and water supply nozzle 25 through the through hole 21b by starting the air and water supply apparatus 27 to supply water. This permits the introductory tube 20 to wash away filth or the like adhering to the observation window member 24.

In this case, the operator depresses the air and water supply push-button switch 28 once. The introductory tube 20 jets air as indicated by an arrow from the opening in the air and water supply nozzle 25 through the through hole 21b by starting the air and water supply apparatus 27 to supply air. This permits the introductory tube 20 to remove water droplets adhering to a surface of the observation window member 24. And, the operator depresses the suction push-button switch 29. The introductory tube 20 sucks body fluid or the like from the opening of the through hole 21c by starting the suction device.

The introductory tube 20 under a rotating condition then passes through, the S-shaped colon portion 73, a bending portion as a boundary between the S-shaped colon portion 73 and colon descendens 74 with low movability, a splenic flexture portion 76 as a boundary between colon descendens 74 and a transverse colon portion 75 with high movability and a liver bending portion 77 as a boundary between the transverse colon portion 75 and colon ascendens 78 and, as shown in FIG. 8, reaches the vicinity of a caecum portion 79 as a target portion.

The operator, after determining that distal end portion of the introductory tube 20 reaches the vicinity of the caecum portion 79 from an endoscope image displayed on a screen of the monitor 6, gives an instruction, for example, to a staff to stop driving of the motor 44. At this time, to perform endoscope checking for the internal large intestine, the operator shifts to pulling-back of the endoscope insertion portion 11 for the checking.

After completion of the checking, the operator removes the endoscope insertion portion 11 from the introductory tube 20, scraps the introductory tube 20 and inserts the endoscope insertion portion 11 into a new introductory tube 20 before use. This permits the endoscope system 1 to perform the next inspection without need of cleaning and sterilizing the endoscope 2.

Accordingly, the introductory tube 20 can reduce a contact resistance occurring between the inner peripheral surface of the spiral tube 23 and the elastic cover tube 21 and, in performing insertion into the body cavity, provides a satisfactory propulsion function, thus facilitating insertion of the endoscope insertion portion 11 into the body cavity.

Insertion of the endoscope insertion portion into the introductory tube 20 surely prevents the endoscope insertion portion 11 from coming into direct contact with the body cavity during the checking. Accordingly, the staff can reuse a combination of the endoscope 2 pulled out of the introductory tube 20 with a new introductory tube 20 without cleaning and sterilizing after completion of the checking. This can relieve the staff of troublesome cleaning and sterilizing of the endoscope 2 and the introductory tube 20 at every checking completion.

In the present embodiment, a large intestine is taken as a tubular body cavity to be inserted with the endoscope insertion portion 11 covered with the introductory tube 20, but a tubular body cavity inserted with the endoscope insertion portion 11 is not limited to the large intestine, but may be any of tubular body cavities such as oral cavity to esophagus, stomach and small intestine.

The rotational direction of the introductory duct 20 in the present embodiment may be only one way (advance direction) or clockwise/counterclockwise rotation may be performed in a fixed cycle or by arbitrary switching. A combination of clockwise and counterclockwise rotations permits the introductory tube 20 to repeat back and forth movement in the body cavity. Even if the distal end of the introductory tube 20 is caught in a small recessed portion or the like in the intestine wall during forward movement, the catch can be relieved during rearward movement. During the second forward movement, the positions of the intestine and the introductory tube 20 are finely dislocated from each other, which permits smooth advance without causing recurrence of the catch.

The friction reduction portion may be configured as shown in FIGS. 9 and 10.

As shown in FIG. 9, between an inner peripheral surface of the spiral tube 23 and the elastic cover tube 21, there are fixed regulating rings 52 constituting a regulating portion with one set of two rings on both sides of each of ring members 51B arranged at predetermined intervals.

As shown in FIG. 10, the regulating rings 52 sandwich the ring member 51B so as to float the ring member 51B from the elastic cover tube 21 by a predetermined distance. This permits the ring member 51B to regulate movement in a longitudinal-axis direction by the regulating ring 52 and to rotate relative to an inner peripheral surface of the spiral tube 23 without contacting the elastic cover tube 21.

Thus, the ring member 51B can reduce a contact resistance against the inner peripheral surface of the spiral tube 23 without causing friction against the elastic cover tube 21.

This enables the introductory tube 20 to reduce a contact resistance generated between the inner peripheral surface of the spiral tube 23 and the elastic cover tube 21 compared with the case in the first embodiment.

The ring member 51B and the regulating ring 52 have biological compatibility, respectively, as described in the first embodiment and are formed of highly slidable material, for example, fluororesin, such as PTFE, or polyethylene or stainless steel.

The ring member 51B and the regulating ring 52 may be constituted using fluororesin coating such as PTFE for improvement of slidability as described in the first embodiment. In addition, the ring member 51B and the regulating ring 52 may be configured using polyacetal POM (Polyoxymethylene) as material.

Hence, the introductory tube 20 prevents the inner peripheral surface of the spiral tube 23 from coming into contact with the outer peripheral surface of the elastic cover tube 21 over the overall length by forming the plurality of ring members 51B and the plurality of regulating rings 52 at predetermined intervals to bring the inner peripheral surface of the spiral tube 23 into contact with the ring member 51B even if the rotating spiral tube 23 is twisted in a winding body-cavity inner tube line. Furthermore, at this time, the introductory tube 20 is set so that a distance between the inner peripheral surface of the spiral tube 23 and an outer peripheral surface of the elastic cover tube 21 is not constant over the range from the distal end side to the proximal end side.

Accordingly, the introductory tube 20 can reduce a contact resistance against the inner peripheral surface of the spiral tube 23 without causing friction against the elastic cover tube 21.

Thus, a contact area between the inner peripheral surface of the spiral tube 23 and the elastic cover tube 21 further decreases, which enables the introductory tube 20 to reduce a contact resistance occurring therebetween.

The friction reduction section may be configured as shown in FIG. 11.

As shown in FIG. 11, between an inner peripheral surface of the spiral tube 23 and the elastic cover tube 21, ring members 53 with circular cross sections to be irregularities as a friction reduction section are bonded, for example, over the range from the distal end side to the proximal end side of an outer peripheral surface of the elastic cover tube 21 at predetermined intervals using a fixing agent such as adhesives. The friction reduction section does not need to be provided within the range of the distal end side to the proximal end side.

Each of the ring members 53, owing to its circular cross section, is configured so as to come into line contact in between the inner peripheral surface of the spiral tube 23 and the elastic cover tube 21. Accordingly, the ring members 53 can reduce a contact resistance between the inner peripheral surface of the spiral tube 23 and the elastic cover tube 21 compared with the case of the ring member 51B.

Since each of the ring members 53 is spherical, generates no edge against the inner peripheral surface of the spiral tube 23 and further can reduce a contact resistance between the inner peripheral surface of the spiral tube 23 and the elastic cover tube 21.

Each of the ring members 53 has biological compatibility, in the same way as described in the first embodiment and are formed of highly slidable material, for example, fluororesin, such as PTFE, or polyethylene or stainless steel. Each of the ring members 53 may be constituted by applying fluororesin coating such as PTFE for improvement of slidability same as described in the first embodiment.

Each of the ring members 53 may be configured using polyacetal (Polyoxymethylene) as material. Moreover, each of the ring members 53 may be constituted of an expandable member such as an O-ring for easy installation.

Each of the ring members 53 may be rotatably configured by fixing one set of two regulating rings 52 on both sides as described above although they are not shown. This permits each of the ring members 53 to further reduce a contact resistance between the inner peripheral surface of the spiral tube 23 and the elastic cover tube 21.

The friction reduction section may be configured as shown in FIG. 12.

As shown in FIG. 12, between the inner peripheral surface of the spiral tube 23 and the elastic cover tube 21, there are formed groove portions 54 as disposal portions over the diametrical direction at a position of the elastic cover tube 21 where the ring members 53 is disposed against the ring members 53 arranged at predetermined intervals.

Fitting the ring members 53 onto the groove portions 54 so as to float from the cover tube 21 by a predetermined distance regulates movement of the ring members 53 in the longitudinal direction, and the ring members 53 are rotatably formed.

This causes the ring member 53 to be regulated to move in a longitudinal-axis direction by the groove portion 54 and to be rotatable relative to an inner peripheral surface of the spiral tube 23 without contacting the elastic cover tube 21. Thus, the ring member 53 can reduce a contact resistance against the inner peripheral surface of the spiral tube 23 without causing friction against the elastic cover tube 21.

This permits each of the ring members 53 to further reduce a contact resistance between the inner peripheral surface of the spiral tube 23 and the elastic cover tube 21.

In the present embodiment, a rotational driving force of the motor 44 is transmitted to the proximal end side of a spiral tube 23 as a rotating cylindrical body to rotate the whole spiral tube 23, but the present invention is not limited to this. For example, the rotational driving force of the motor 44 may be transmitted to the middle portion of the spiral tube 23 to rotate the whole spiral tube 23. Otherwise, the rotational driving force may be transmitted to the distal end portion of the spiral tube 23 to rotate the whole spiral tube 23.

In the present embodiment, the present invention is applied to a configuration of a disposable sheath as the introductory tube 20, but the present invention is not limited to this. Naturally, the present invention may be applied to a type formed integrally with an endoscope insertion portion as an introductory tube and, what is called, an over-tube for endoscope as a tubular-shaped tube formed so as to be harder than a flexible tube portion of an endoscope (hereinafter referred to as “over-tube”). It is sufficient that the friction reduction section can reduce a contact resistance between an inner peripheral surface of the spiral tube 23 and a non-rotation portion and significant propulsion function can be obtained.

### Second Embodiment

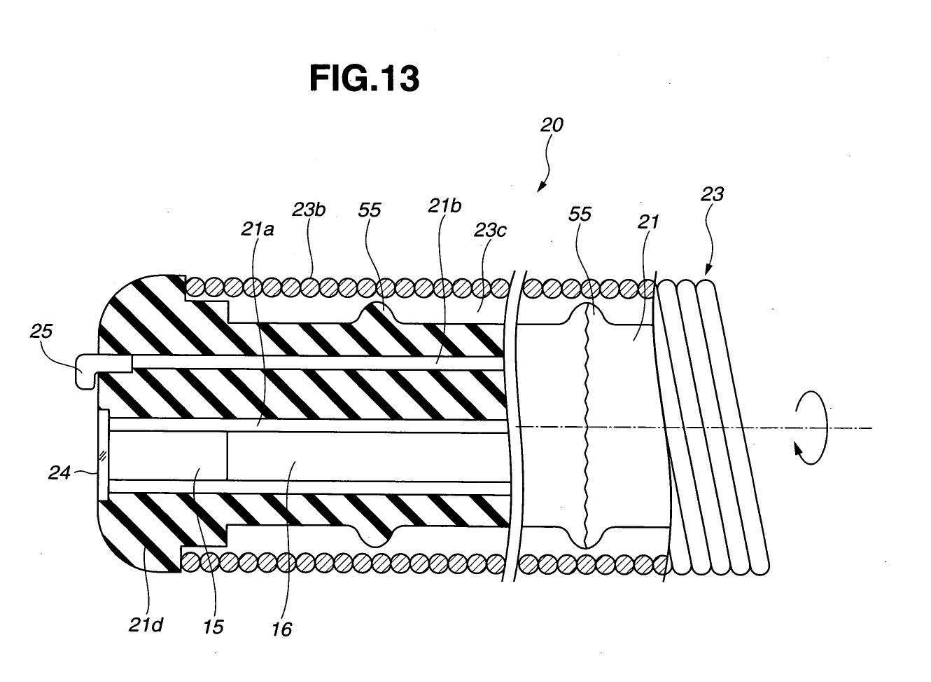

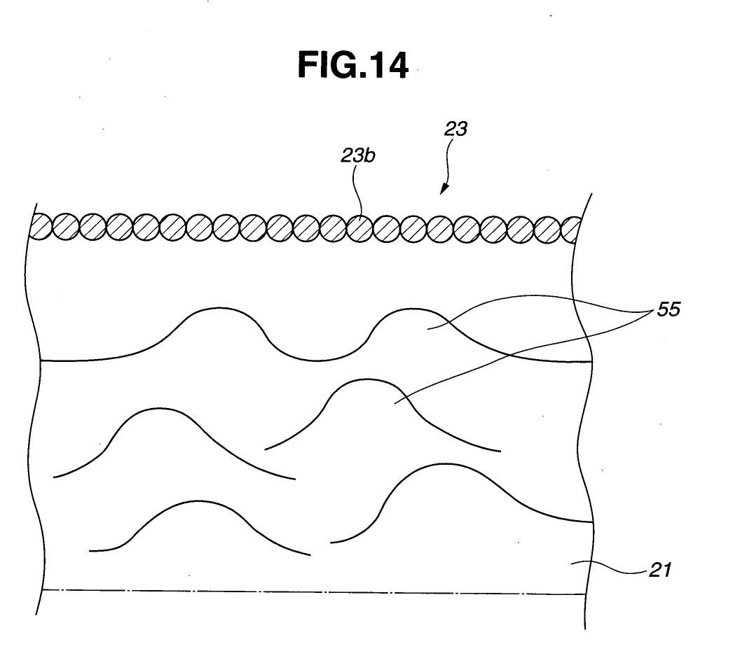

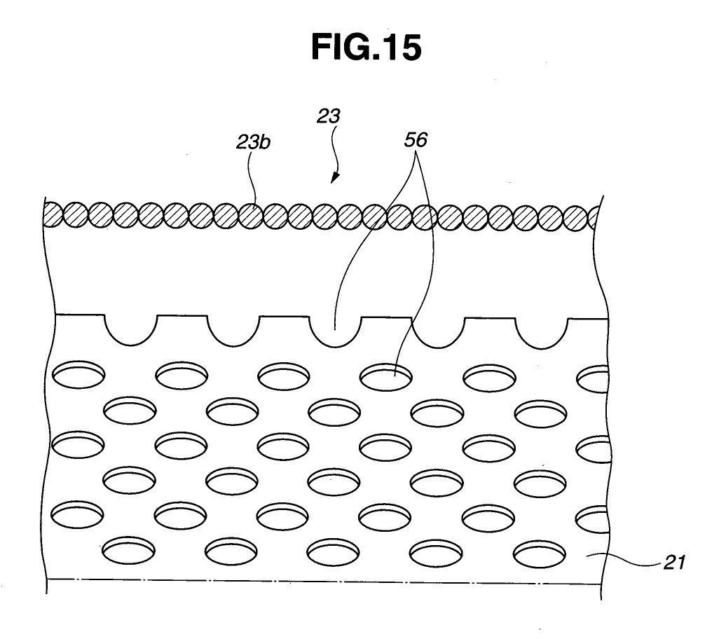

FIGS. 13 to 15 relate to a second embodiment according to the present invention. FIG. 13 is a descriptive view of a substantial part showing the vicinity of a distal end portion of an introductory tube configuring an endoscope system in a second embodiment, FIG. 14 is an enlarged view of a substantial part showing a first variant in FIG. 13 and FIG. 15 is an enlarged view of a substantial part showing a second variant in FIG. 13.

The first embodiment comprises ring members to be irregularities as a friction reduction section between an inner peripheral surface of the spiral tube 23 and the elastic cover tube 21 so as to reduce a contact resistance therebetween, while a second embodiment is configured so as to change the shape of the elastic cover tube 21 for obtaining irregularities as a friction reduction section. Other configurations are the same as in the first embodiment and descriptions thereof are omitted and the same configurations have the same symbols for description.

As shown in FIG. 13, an introductory tube 20 of the second embodiment is configured so that the elastic cover tube 21 has a plurality of protrusions 55 provided over the range from the distal end to the proximal end at predetermined intervals in the longitudinal direction so as to be irregularities as a friction reduction section.

Thus, the introductory tube 20, having the plurality of protrusions 55 provided over the range from the distal end to the proximal end at predetermined intervals on an outer peripheral surface of the elastic cover tube 21, prevents an inner peripheral surface of the spiral tube 23 and the outer peripheral surface of the elastic cover tube 21 from coming into contact with each other over the whole length by bringing the inner peripheral surface of the spiral tube 23 into contact with the plurality of protrusions 55 even if the spiral tube 23 under a rotating condition gets twisted in a winding body-cavity inner tube line. Furthermore, at this time, the introductory tube 20 is set so that a distance between the inner peripheral surface of the spiral tube 23 and an outer peripheral surface of the elastic cover tube 21 is not constant over the range from the distal end side to the proximal end side.

Accordingly, the introductory tube 20 can reduce a contact resistance occurring between the inner peripheral surface of the spiral tube 23 and the outer peripheral surface of the elastic cover tube 21 in the same way as in the first embodiment. Moreover, each of the protrusions 55, having almost the same spherical shape as the ring members 53, comes into line contact to further reduce a contact resistance.

The protrusions 55 are provided at predetermined intervals in the longitudinal-axis direction, but may be provided irregularly on a surface of the elastic cover tube 21 as shown in FIG. 14. Further, as shown in FIG. 15, holes 56 may be irregularly formed in a surface of the elastic cover tube 21 in place of the protrusions 55.

In the present embodiment, the present invention is applied to a configuration of a disposable sheath as the introductory tube 20, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. It is sufficient that the friction reduction section can reduce a contact resistance between an inner peripheral surface of the spiral tube 23 and a non-rotation portion and a significant propulsion function can be obtained.

### Third Embodiment

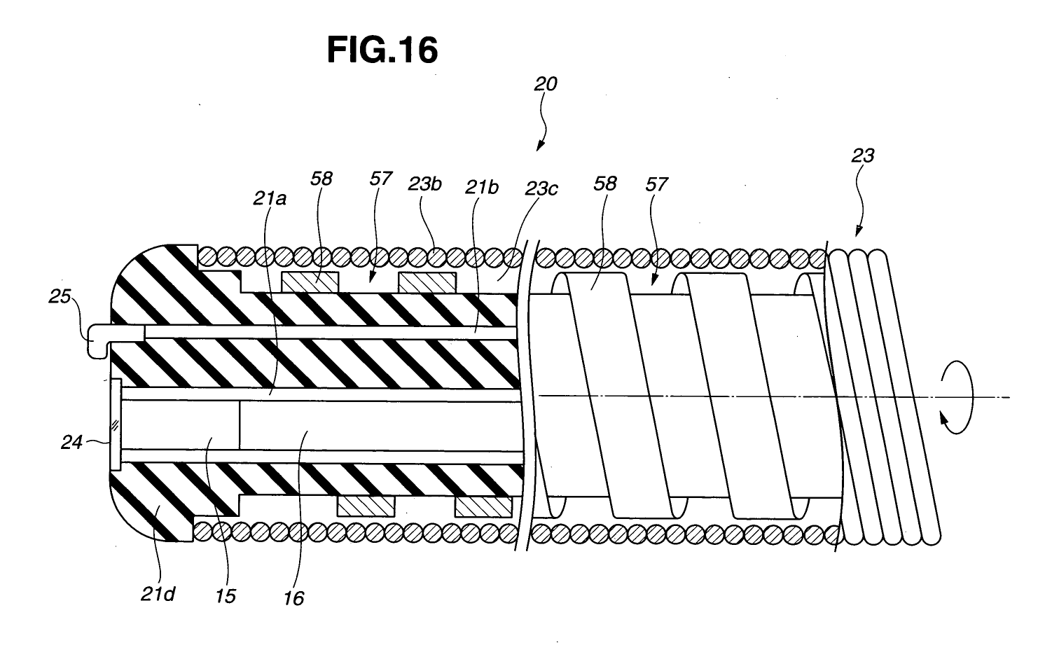

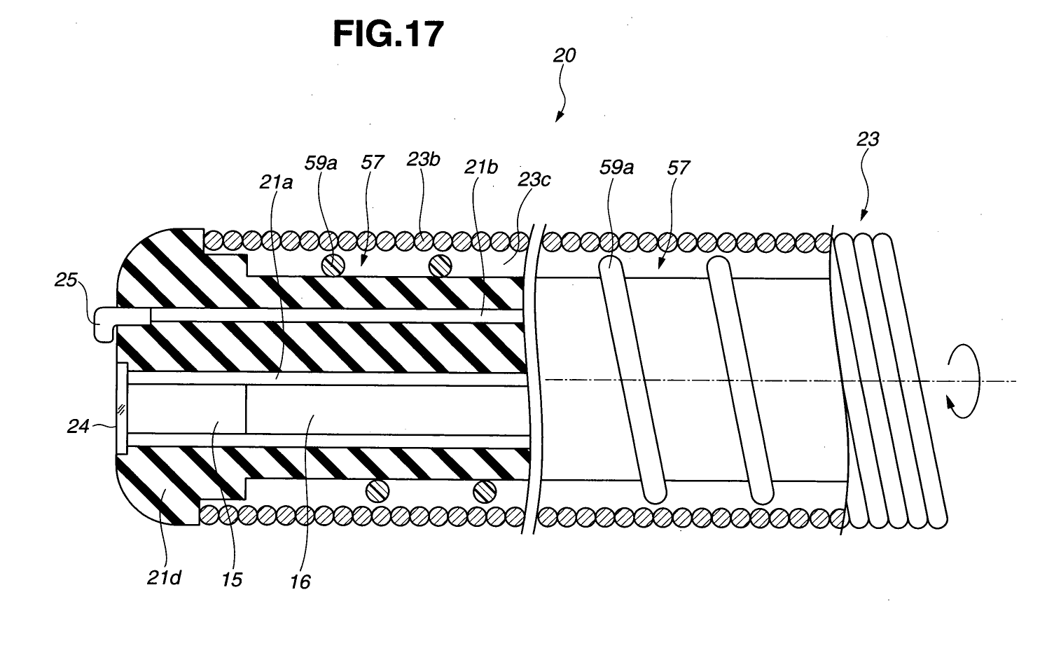

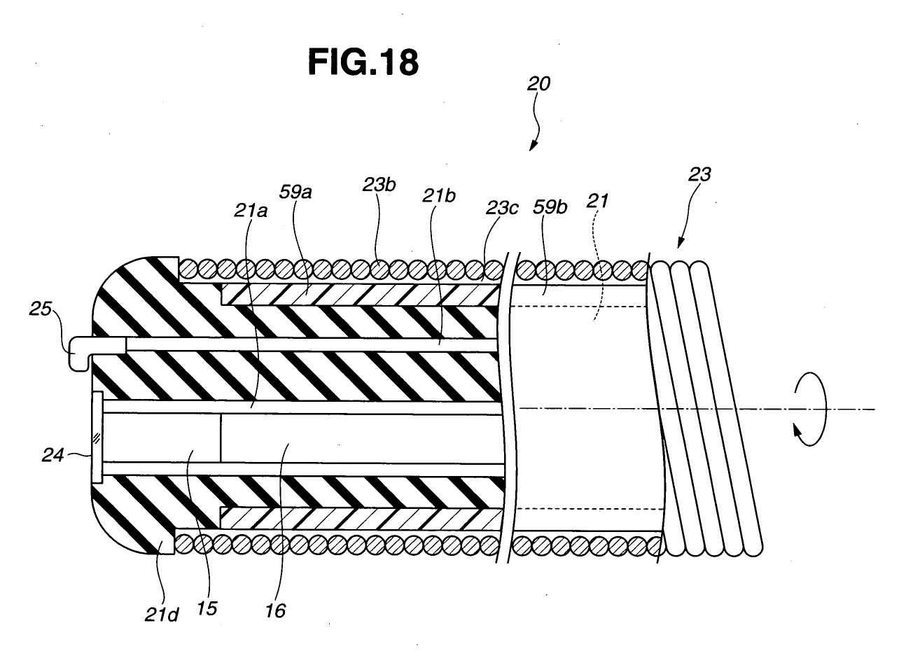

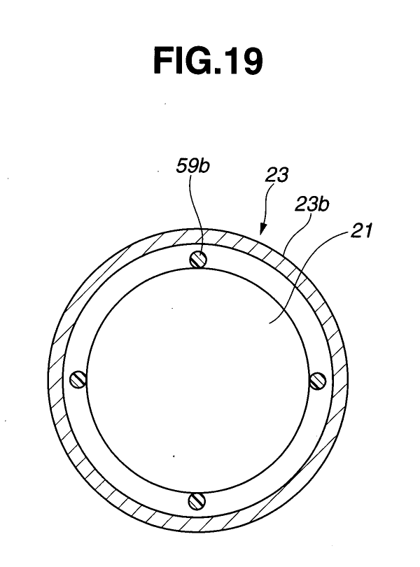

FIGS. 16 to 19 relate to a third embodiment according to the present invention. FIG. 16 is a descriptive view of a substantial part showing the vicinity of a distal end portion of an introductory tube configuring an endoscope system in a third embodiment, FIG. 17 is a descriptive view of a substantial part showing a first variant in FIG. 16, FIG. 18 is a descriptive view of a substantial part showing the vicinity of a distal end portion of an introductory tube of a second variant in FIG. 16 and FIG. 19 is a sectional view of the substantial part in FIG. 18.

The first embodiment comprises ring members to be irregularities as a friction reduction section between the inner peripheral surface of the spiral tube 23 and the outer peripheral surface of the elastic cover tube 21 so as to reduce a contact resistance therebetween, while a third embodiment is configured so as to provide winding portions at the elastic cover tube 21 for obtaining irregularities as a friction reduction section. Other configurations are the same as in the first embodiment and descriptions thereof are omitted and the same configurations have the same symbols for description.

As shown in FIG. 16, the introductory tube 20 in the third embodiment has winding portions 57 formed over the range of the distal end to the proximal end so as to have irregularities on the elastic cover tube 21 as friction reduction portions. More specifically, each of the winding portions 57 is configured by winding a plate-shaped spiral band 58 around the elastic cover tube 21 at predetermined pitches in the same winding direction as the spiral-shaped portion 23b over the range of the distal end to the proximal end side.

The plate-shaped spiral band 58 is constituted of a material which has biological compatibility and high slidability, for example, fluororesin such as PTFE, polyethylene or stainless steel. The plate-shaped spiral band 58 may be constituted by applying coating of fluororesin such as PTFE for slidability.

Thus, the introductory tube 20, having the winding portion 57 provided over the range from the distal end to the proximal end at predetermined pitches with the plate-shaped spiral band 58 wound and fixed on the elastic cover tube 21, prevents an inner peripheral surface of the spiral tube 23 and outer peripheral surface of the elastic cover tube 21 from coming into contact with each other over the whole length by bringing the inner peripheral surface of the spiral tube 23 into contact with the winding portion 57 even if the spiral tube 23 under a rotating condition gets twisted in a winding body-cavity inner tube line. Furthermore, at this time, the introductory tube 20 is set so that a distance between the inner peripheral surface of the spiral tube 23 and an outer peripheral surface of the elastic cover tube 21 is not constant over the range from the distal end side to the proximal end side.

Accordingly, the introductory tube 20 can reduce a contact resistance occurring between the inner peripheral surface of the spiral tube 23 and the outer peripheral surface of the elastic cover tube 21 in the same way as in the first embodiment.

The winding portion 57 may be constituted by spirally winding a tube around the elastic cover tube 21 as shown in FIG. 17 in place of the plate-shaped spiral band 58.

As shown in FIG. 17, the winding portion 57 is constituted by winding and fixing a tube 59a around the elastic cover tube 21 at predetermined pitches in the same winding direction as for the spiral-shaped portion 23b over the range from the distal end to the proximal end side.

The tube 59a is constituted of a material which has biological compatibility and high slidability, for example, fluororesin such as PTFE, polyethylene or stainless. The tube 59a may be constituted by applying coating of fluororesin such as PTFE for high slidability.

Thus, the introductory tube 20, having the winding portions 57 formed by winding the tube 59a around the elastic cover tube 21 at predetermined pitches over the range from the distal end to the proximal end side, reduces a contact area between the inner peripheral surface of the spiral tube 23 and the elastic cover tube 21, thus reducing a contact resistance occurring therebetween.

The tube may be fixed on the elastic cover tube 21 over the longitudinal-axis direction as shown in FIG. 18 in place of winding configuration.

As shown in FIG. 18, tubes 59b are fixed on the elastic cover tube 21 over the range from the distal end to the proximal end side in the longitudinal-axis direction. The plurality of tubes 59b are provided, as shown in FIG. 19, in diametrical directions of the elastic cover tube 21. The four tubes 59b are provided in FIG. 19 and any number of the tubes is permissible if more than one. Thus, the introductory tube 20 is constituted only by fixing the tube 59b in the longitudinal-axis direction, which provides easier manufacture.

In the present embodiment, the present invention is applied to a configuration of a disposable sheath as the introductory tube 20, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. It is sufficient that the friction reduction section reduces a contact resistance between an inner peripheral surface of the spiral tube 23 and a non-rotation portion and provides a significant propulsion function.

### Fourth Embodiment

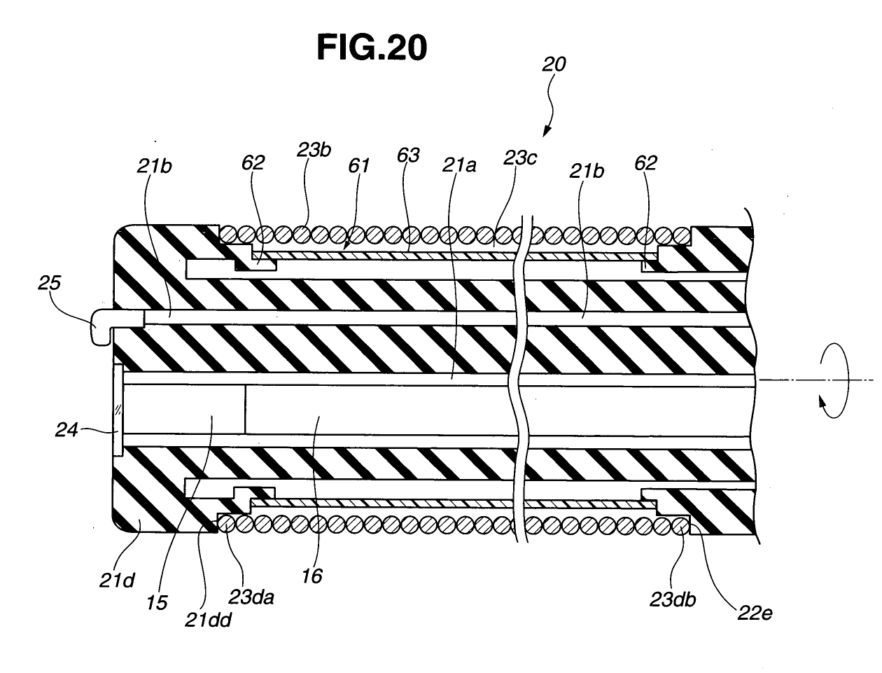

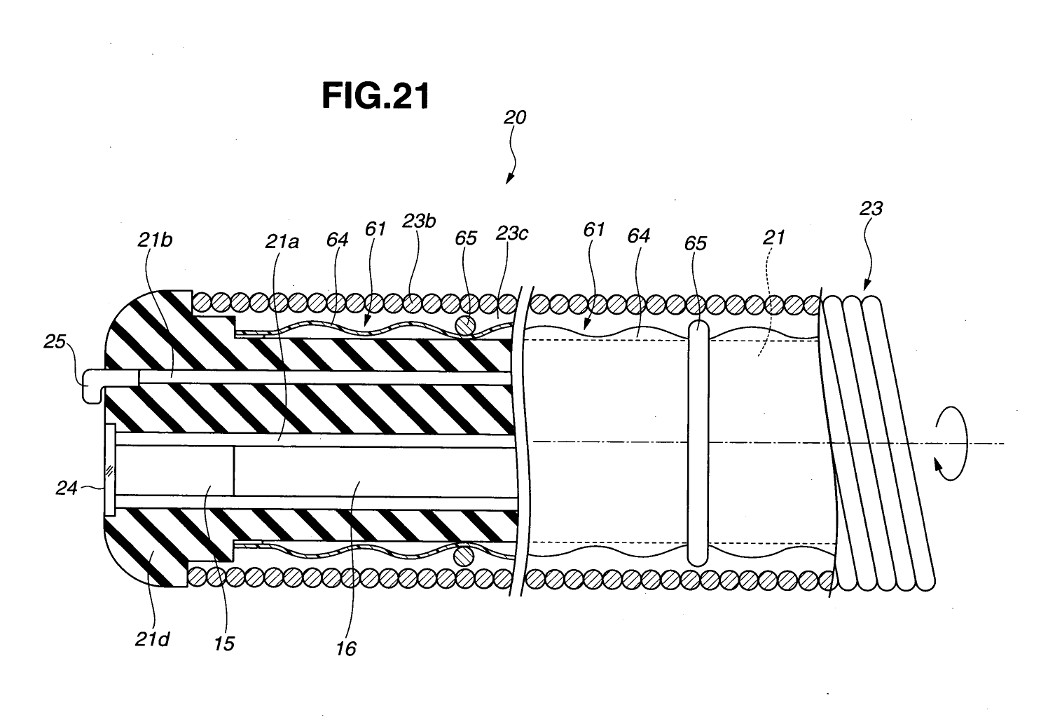

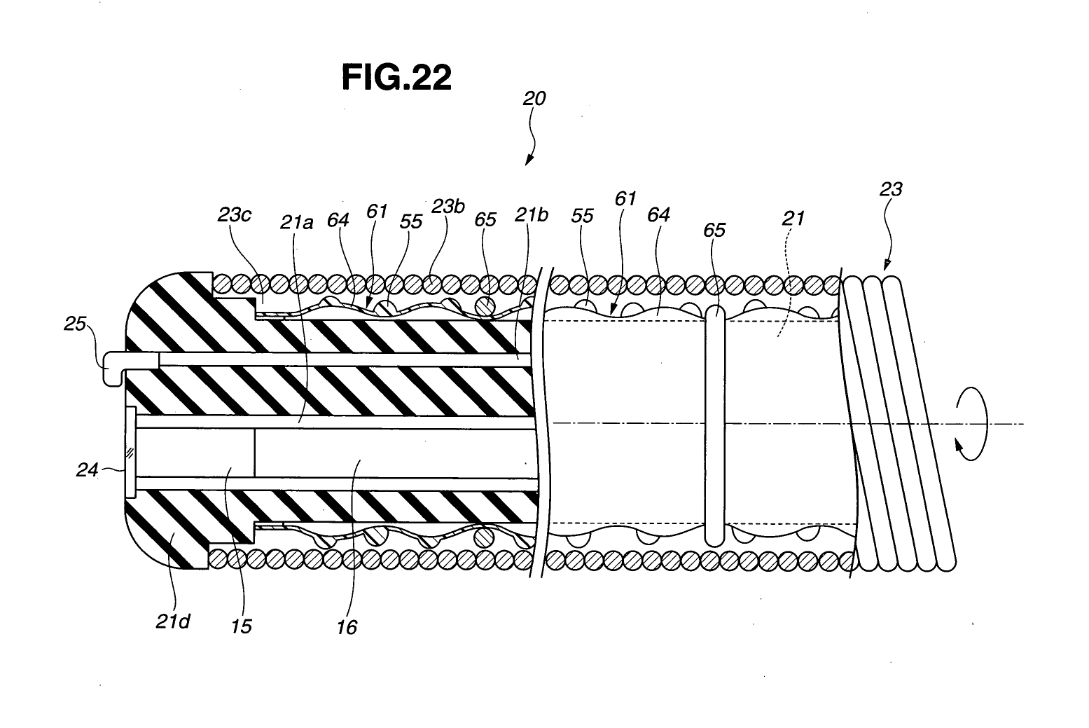

FIGS. 20 to 22 are views of a fourth embodiment according to the present invention. FIG. 20 is a descriptive view of a substantial part showing the vicinity of a distal end portion of an introductory tube configuring an endoscope system in a fourth embodiment, FIG. 21 is a descriptive view of a substantial part showing the vicinity of the distal end portion of an introductory tube of a first variant in FIG. 20, FIG. 22 is a descriptive view of a substantial part showing the vicinity of the distal end portion of an introductory tube of a second variant in FIG. 20.

The first embodiment comprises ring members to be irregularities as a friction reduction section between the inner peripheral surface of the spiral tube 23 and the outer peripheral surface of the elastic cover tube 21 so as to reduce a contact resistance therebetween, while a fourth embodiment is configured so as to provide a covering portion at the elastic cover tube 21 for obtaining irregularities as a friction reduction section. Other configurations are the same as in the first embodiment and descriptions thereof are omitted and the same configurations have the same characters for description.

As shown in FIG. 20, the introductory tube 20 in the fourth embodiment has a covering portion 61 formed on the elastic cover tube 21 over the range from the distal end to, the proximal end side as a friction reduction portion. More specifically, the covering portion 61 has a covering tube 63 disposed rotatably around the longitudinal axis over the range from the distal end to the proximal end side so as to prevent break-off on a protrusion portion 62 provided on the elastic cover tube 21.

The covering tube 63 is constituted of a material which has biological compatibility and high slidability, for example, fluororesin such as PTFE, polyethylene or stainless. The covering tube 63 may be constituted by applying coating of fluororesin such as PTFE for high slidability. A rotational relationship between the spiral-shaped portion 23b, the covering tube 63 and the elastic cover tube 21 is, for example, 100 rpm/min.>20 rpm/min.>0 rpm/min.

Thus, the introductory tube 20, having the covering tube 63 as the covering portion 61 on the elastic cover tube 21 over the range from the distal end to the proximal end side, prevents the inner peripheral surface of the spiral tube 23 from coming into direct contact with the outer peripheral surface of the elastic cover tube 21. The introductory tube 20 can reduce a contact resistance by rotating the covering tube 63 even if the covering tube 63 receives a contact resistance.

Hence, the introductory tube 20 prevents the inner peripheral surface of the spiral tube 23 from coming into contact with the outer peripheral surface of the elastic cover tube 21 over the whole length by bringing the inner peripheral surface of the spiral tube 23 into contact with the covering tube 63 even if the spiral tube 23 under a rotating condition gets twisted in a winding body-cavity inner tube line. Furthermore, at this time, the introductory tube 20 is set so that a distance between the inner peripheral surface of the spiral tube 23 and the outer peripheral surface of the elastic cover tube 21 is not constant over the range from the distal end to the proximal end side. Accordingly, the introductory tube 20 can reduce a contact resistance occurring between the inner peripheral surface of the spiral tube 23 and the outer peripheral surface of the elastic cover tube 21 in the same way as in the first embodiment.

The covering portion 61 may be constituted by attaching a bellows type cover member 64 not in a free rotation condition to the elastic cover tube 21 as shown in FIG. 21.

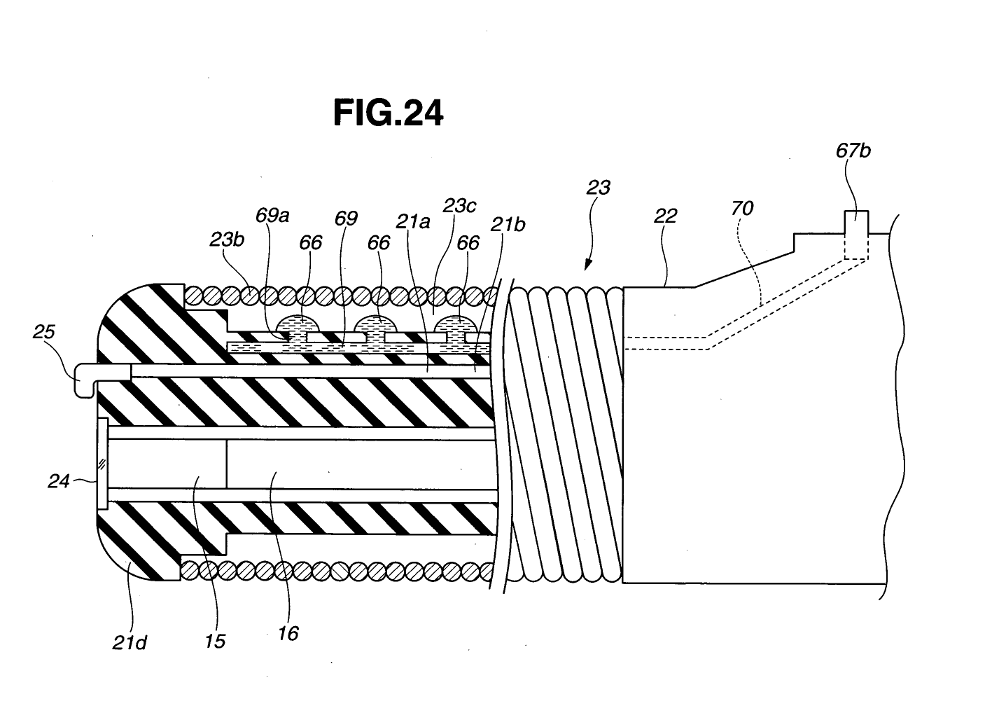

As shown in FIG. 21, the covering portion 61 may be constituted by providing the cover member 64 which is not rotatable and bellows-shaped onto the elastic cover tube 21. As shown in FIG. 24, the covering portion 61 is constituted by attaching the cover member 64 to the outer peripheral surface of the elastic cover tube 21 over the range from the distal end to the proximal end side. Moreover, in the covering portion 61, a looseness prevention ring 65 is provided at a predetermined interval not in close contact with the elastic cover tube 21 by looseness of the cover member 64. The cover member 64 is constituted of a material with biological compatibility and high slidability, for example, fluororesin such as PTFE, polyethylene or stainless. The cover member 64 may be constituted by applying coating of fluororesin such as PTFE for high slidability.

Thus, the introductory tube 20, having the cover member 64 as the covering portion 61 on the outer peripheral surface of the elastic cover tube 21 over the range from the distal end to the proximal end side, prevents the inner peripheral surface of the spiral tube 23 from coming into direct contact with the outer peripheral surface of the elastic cover tube 21.

The introductory tube 20, because the cover member 64 is loosened to a degree that it is not in contact with the elastic cover tube 21, can reduce a contact resistance by the inner peripheral surface of the spiral tube 23.

As shown in FIG. 22, the cover member 64 may be constituted by providing the protrusion portion 55 described above between the inner periphery of the spiral-shaped portion 23b. This permits the cover member 64 to further reduce a contact resistance against the inner periphery of the spiral-shaped portion 23b.

In the present embodiment, the present invention is applied to a configuration of a disposable sheath as the introductory tube 20, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. The friction reduction portion may be one that can reduce a contact resistance between the inner peripheral surface of the spiral tube 23 and the non-rotating portion and provide a sufficient propulsion function.

### Fifth Embodiment

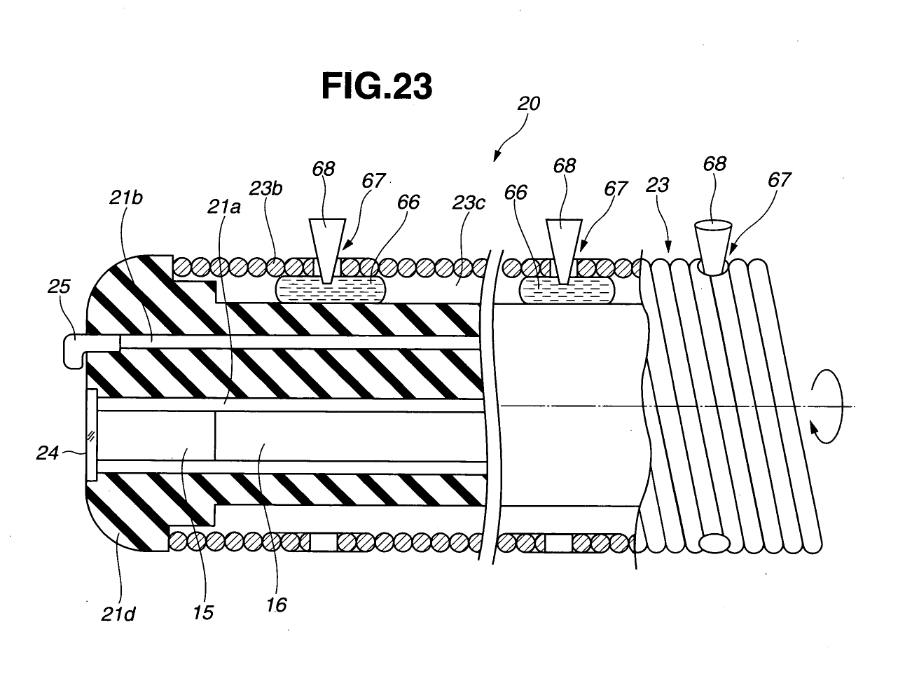

FIGS. 23 and 24 are views of a fifth embodiment according to the present invention. FIG. 23 is a descriptive view of a substantial part showing the vicinity of a distal end portion of an introductory tube configuring an endoscope system in a fifth embodiment and FIG. 24 is a descriptive view of a substantial part showing the vicinity of the distal end portion of an introductory tube of a variant in FIG. 23.

The first embodiment comprises ring members to be irregularities as a friction reduction section between the inner peripheral surface of the spiral tube 23 and the outer peripheral surface of the elastic cover tube 21 so as to reduce a contact resistance therebetween, while a fifth embodiment is configured so as to charge lubricant into between the inner peripheral surface of the spiral tube 23 and the outer peripheral surface of the elastic cover tube 21 as a friction reduction section. Other configurations are the same as in the first embodiment and descriptions thereof are omitted and the same configurations have the same characters for description.

As shown in FIG. 23, the introductory tube 20 in the fifth embodiment is configured so as to charge lubricant 66 into between the inner peripheral surface of the spiral tube 23 and the outer peripheral surface of the elastic cover tube 21 as a friction reduction section. More specifically, the introductory tube 20 is formed with a plurality of lubricant filler holes 67 for charging the lubricant 66 in the spiral-shaped portion 23b at predetermined intervals over the range from the distal end to the proximal end side. A reference character 68 denotes a filler tool for inserting into the lubricant filler hole 67 to charge the lubricant 66.

Accordingly, the introductory tube 20 can charge the lubricant 66 into between the inner peripheral surface of the spiral tube 23 and the outer peripheral surface of the elastic cover tube 21 by charging the lubricant 66 from the lubricant filler hole 67, thus reducing a contact resistance between the inner peripheral surface of the spiral tube 23 and the outer peripheral surface of the elastic cover tube 21 with the lubricant 66.

As shown in FIG. 24, the lubricant 66 may be charged into a lubricant insertion path formed in the elastic cover tube 21 in place of formation of the lubricant filler hole 67 in the spiral-shaped portion 23b.

As shown in FIG. 24, the introductory tube 20 is formed with the lubricant insertion path 69 provided in the elastic cover tube 21 and a communication path 70 communicating with the lubricant insertion path 69 provided in the proximal-end-side component 22. The communication path 70 is formed with the lubricant filler hole 67b on the proximal end side. The lubricant insertion path 69 is formed with a plurality of holes 69a provided at predetermined positions between the inner peripheral surface of the spiral tube 23 and the elastic cover tube 21 at predetermined intervals over the range from the distal end to the proximal end side, and the lubricant 66 jets from the opening 69a. The number of the lubricant insertion passages 69 or the communication paths 70 and the lubricant filler holes 67b may be single or plural.

Accordingly, the introductory tube 20 permits the lubricant 66 to be charged into between the inner peripheral surface of the spiral tube 23 and the outer peripheral surface of the elastic cover tube 21 through the communication path 70 and the lubricant insertion path 69 by charging the lubricant 66 from the lubricant filler hole 67b. Thus, the introductory tube 20 can reduce a contact resistance between the inner peripheral surface of the spiral tube 23 and the outer peripheral surface of the elastic cover tube 21.

In the present embodiment, the present invention is applied to a configuration of a disposable sheath as the introductory tube 20, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. It is sufficient that the friction reduction section reduces a contact resistance between an inner peripheral surface of the spiral tube 23 and a non-rotation portion and provides a significant propulsion function.

### Sixth Embodiment

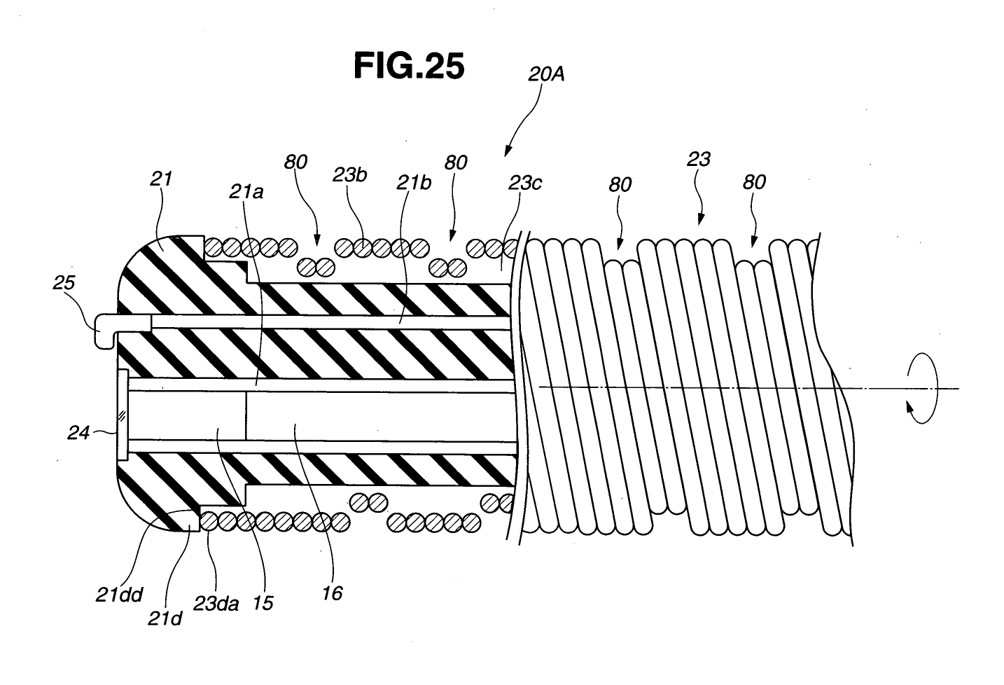

FIG. 25 is a descriptive view of a substantial part showing the vicinity of a distal end portion of an introductory tube configuring an endoscope system in a sixth embodiment according to the present invention.

The first to fifth embodiments are respectively formed with a structure, as a friction reduction section, such as the ring member 51 separate from the spiral tube 23 between the inner peripheral surface of the spiral tube 23 and the outer peripheral surface of the elastic cover tube 21, while a sixth embodiment is configured so as to have an inner protrusion portion, as a friction reduction section having irregularities, formed by protruding a part of the spiral tube 23 to the internal diameter side. Other configurations are the same as in the first embodiment and descriptions thereof are omitted and the same configurations have the same characters for description.

As shown in FIG. 25, an introductory tube 20A in a sixth embodiment has a plurality of inner protrusion portions formed by protruding a part of the spiral tube 23 to an inside diameter side over the range from the distal end to the proximal end side as a friction reduction portion having irregularities. More specifically, the spiral tube 23 is formed with a plurality of valley portions 80 formed over the range from the distal end to the proximal end side by winding a part of metal strand constituting the spiral-shaped portion 23b at substantially uniform intervals, for example, by applying 2 turns after every 5 turns to position to the inner periphery side.

Thus, the introductory tube 20A, having the plurality of valley portions 80 provided over the range from the distal end to the proximal end side at predetermined intervals on an outer peripheral surface of the elastic cover tube 21, prevents an inner peripheral surface of the spiral tube 23 and the outer peripheral surface of the elastic cover tube 21 from coming into contact with each other over the whole length by bringing the inner peripheral surface of the spiral tube 23 into contact with the plurality of valley portions 80 even if the spiral tube 23 under a rotating condition gets twisted in a winding body-cavity inner tube line. Furthermore, the introductory tube 20A is set so that a distance between the inner peripheral surface of the spiral tube 23 and the outer peripheral surface of the elastic cover tube 21 is not constant over the range from the distal end to the proximal end side.

Accordingly, the introductory tube 20A can reduce a contact resistance occurring between the inner peripheral surface of the spiral tube 23 and the outer peripheral surface of the elastic cover tube 21 in the same way as for the first embodiment.

The introductory tube 20A, having no troublesome work, which requires assembling, as a friction reduction section, a structure such as the ring member 51 separate from the spiral tube 23 described in the first to fifth embodiments, or uniform application of lubricant onto the overall length of a long tube. This permits high workability and productivity and prevention of softness degradation of the whole introductory tube due to poor bendability in inserting caused by the structure. Furthermore, the introductory tube 20A, having a spiral groove with large head drop on the outer periphery of the spiral tube 23, enhances gripping against the intestine and thus produces high propulsive force.

In the present embodiment, the present invention is applied to a configuration of a disposable sheath as the introductory tube 20A, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. It is sufficient that the friction reduction section reduces a contact resistance between an inner peripheral surface of the spiral tube 23 and a non-rotation portion and provides a significant propulsion function.

### Seventh Embodiment

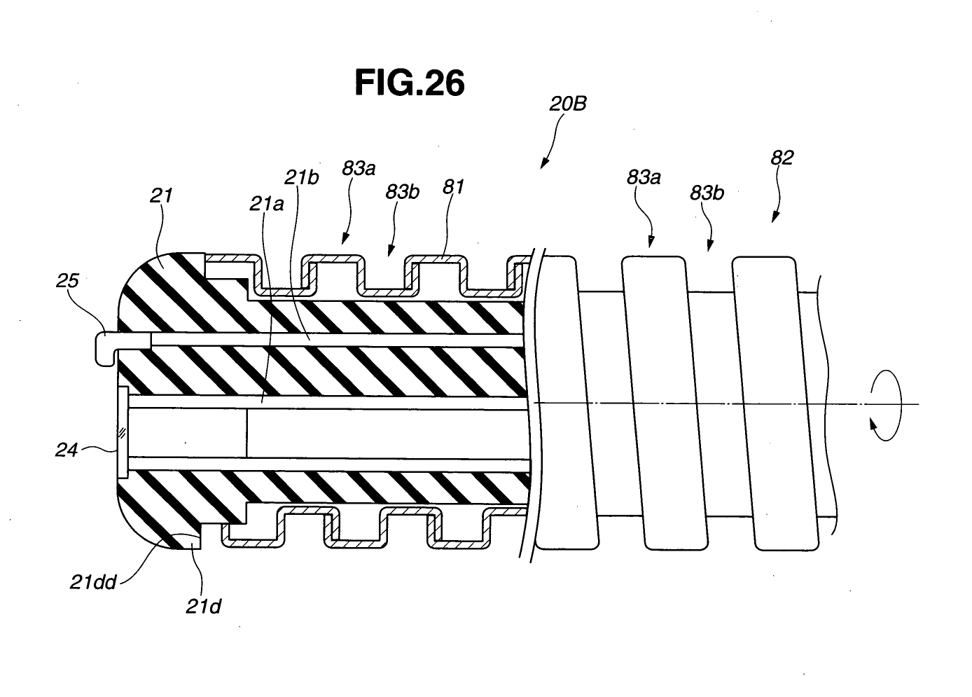

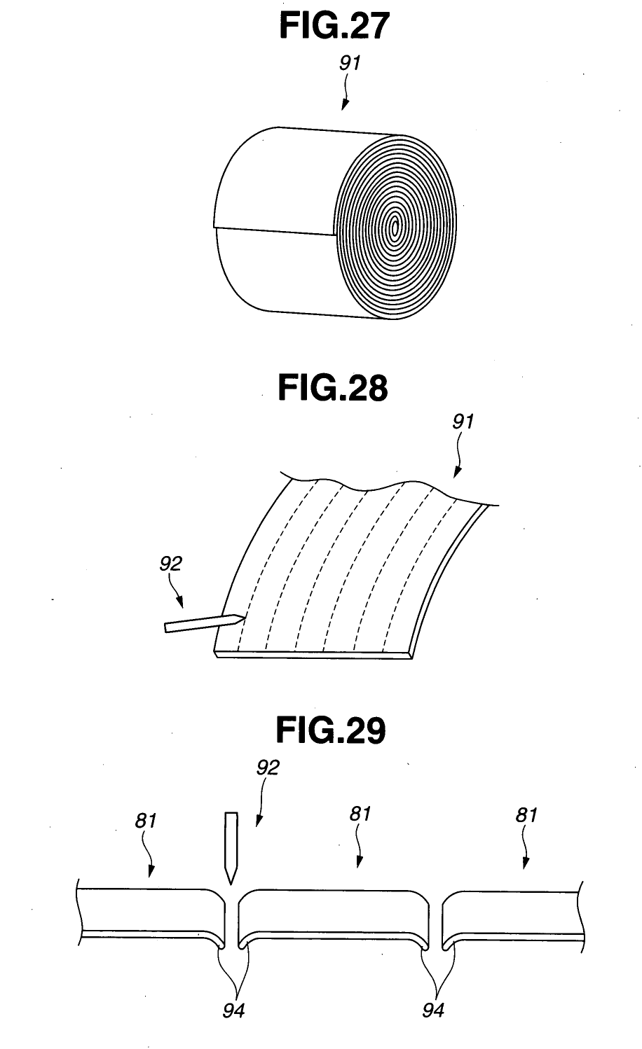

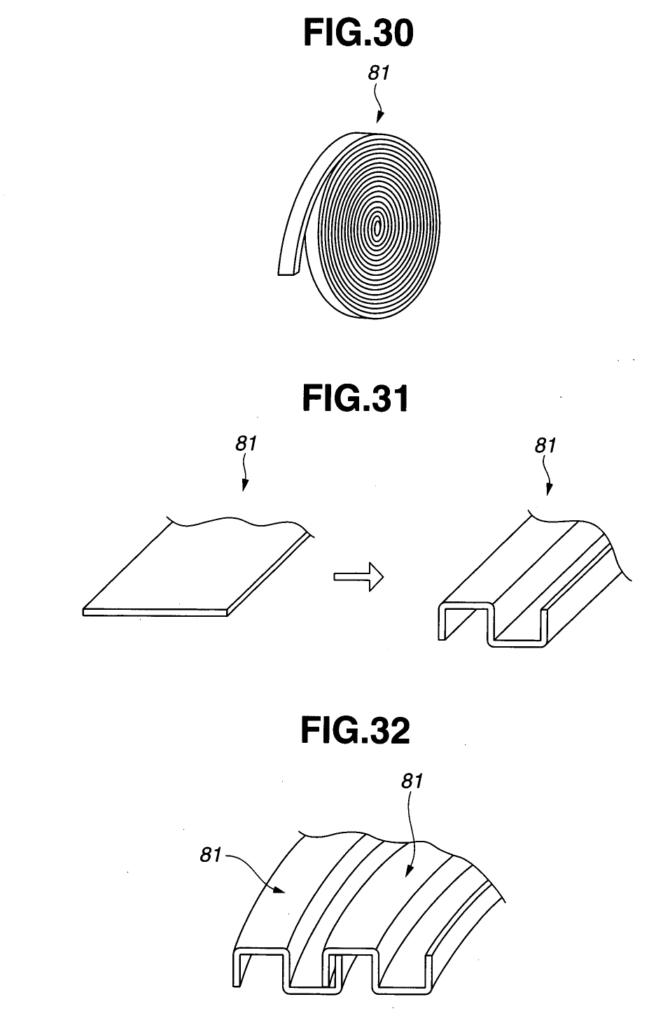

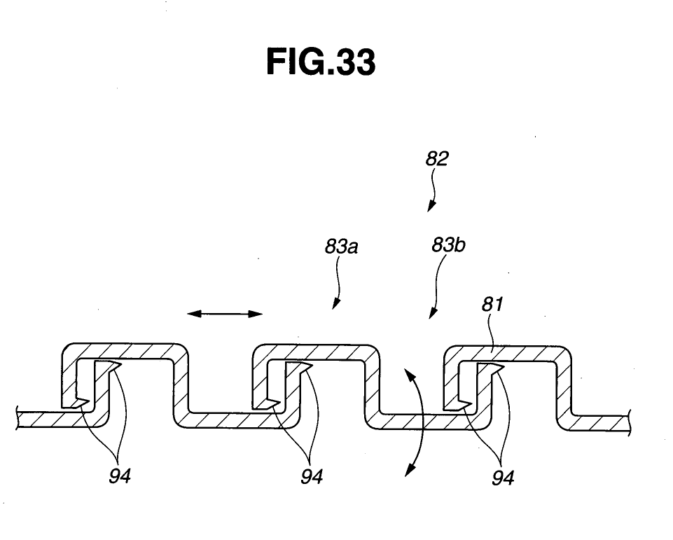

FIGS. 26 to 33 are views of a seventh embodiment according to the present invention. FIG. 26 is a descriptive view of a substantial part showing the vicinity of a distal end portion of an introductory tube configuring an endoscope system in a seventh embodiment, FIG. 27 is an external perspective view showing a broad metal sheet as a raw material of a plate-shaped metal member, FIG. 28 is an outline view showing such a state that the metal sheet in FIG. 27 is cut to plate-shaped metal members with small width using a cutter, FIG. 29 is an outline view showing such a state that burring is generated on plate-shaped metal members in a cutting operation in FIG. 28, FIG. 30 is an external perspective view showing plate-shaped metal members formed in cutting operations in FIGS. 28 and 29, FIG. 31 is a descriptive view in plastically deforming plate-shaped metal members in FIG. 30 using a metal mold, FIG. 32 is an outlined perspective view showing part of a spiral tube formed by engaging a plastically deformed plate-shaped metal members with other plate-shaped metal members and spirally winding them around a core member not shown, and FIG. 33 is an outlined sectional view showing an operation of the spiral tube formed in FIG. 32.

The sixth embodiment is configured by forming a plurality of valley portions as inner protrusion portions so as to form irregularities over the range from the distal end to the proximal end side by applying a part of metal strand generally equidistantly to the inside periphery against a closely wound coil formed so as to have predetermined flexibility by spirally winding the metal strand of a predetermined diameter as the spiral tube 23, while the seventh embodiment is configured so as to have a spiral tube formed with a plurality of valley portions as inner protrusion portions to have irregularities over the range from the distal end to the proximal end side using a plate-shaped metal member. Other configurations are the same as in the sixth embodiment and descriptions thereof are omitted and the same configurations have the same characters for description.

As shown in FIG. 26, an introductory tube 20B in a seventh embodiment has a plurality of valley portions 83b formed by protruding a part of the spiral tube 82 to an inside diameter side over the range from the distal end to the proximal end side as a friction reduction portion having irregularities. More specifically, the spiral tube 82 is constituted of a plate-shaped metal member (metal tape) 81, and formed into a long shape and further into an uneven shape so as to have irregularities as a friction reduction portion.

The spiral tube 82, after the plate-shaped metal member 81 is plastically deformed into an uneven shape, is formed so as to be extendable by spiral winding so that adjacent members engage with each other. The spiral tube 82 is formed so that the width of a crest portion 83a is larger than that of a valley portion 83b. The spiral tube 82 is formed into 0.5 or more in R shape of a corner of the crest portion 83a to prevent damage in a body cavity.

Preferably, the plate-shaped metal member 81 has a board width of, for example, 6 mm or less in order to secure the flexibility of the introductory tube 20B. The plate-shaped metal member 81 is formed so that burring faces inward for prevention of damage in a body cavity.

The plate-shaped metal member 81 is formed with a crest portion 83a and a valley portion 83b. Engagement of one end serving as a valley portion 83b with one end serving as the next crest portion 83a forms the spiral tube 82 into a long shape. Accordingly, on an outer surface of the spiral tube 82, a spiral-shaped portion is produced.

Referring now to FIGS. 27 to 32, a more detailed manufacturing method for the spiral tube 82 will be described later.

First, as shown in FIG. 27, the plate-shaped metal member 81 uses a wide metal sheet 91 as a raw material. The metal sheet 91 is cut to a plurality of narrow plate-shaped metal members 81 with a cutter 92 or the like, as shown in FIG. 28.

At this time, the plate-shaped metal member 81 to be formed is cut with a cutter 92 pressed thereagainst from one side of the metal sheet 91 as shown in FIG. 29, so that burring 94 is generated so as to face inward. The plate-shaped metal member 81 is formed so as to be narrow as shown in FIG. 30.

Next, the plate-shaped metal member 81 formed so as to be narrow is plastically deformed into an uneven shape with a metal mold (not shown) as shown in FIG. 31. The plastically-deformed plate-shaped metal member 81 is combined with other plate-shaped metal member 81 plastically-deformed as shown in FIG. 32, and the spiral tube 82 is formed by spirally winding around a core member (not shown). The plastically-deformed plate-shaped metal member 81 is spirally wound for formation in mutual engagement with other plastically-deformed plate-shaped metal member 81.

On the spiral tube 82, burring 94 will not protrude to the body-cavity inner tube line side, facing inward, as shown in FIG. 33. The spiral tube 82 is expandable and bendable as the result of mutual movement of the respective plate-shaped metal members 81 in engagement. Accordingly, the spiral tube 82, forming the respective plate-shaped metal members 81 in mutual engagement, requires no bonding and generates no stiffness due to bonding.

The spiral tube 82 formed in this way, after being cleaned for prevention of coloring due to heat treatment, is incorporated into a continuous furnace for heat treatment. The continuous furnace is capable of easily heat-treating the long spiral tubes 82. The heat treatment is performed at 300 to 350° C. for 5 to 15 minutes for coloring prevention and high torque followability, which provides heating without attention to coloring and satisfactory annealing. Furthermore, after the heat treatment, the spiral tube 82 is subjected to chemical polishing on an outer surface thereof. The chemical polishing is implemented for high slidability and removal of burring 94.

The introductory tube 20B prevents an inner peripheral surface of the spiral tube 82 and an outer peripheral surface of the elastic cover tube 21 from contacting each other over the full length by bringing the valley portion 83b of the spiral tube 82 into contact with an outer peripheral surface of the elastic cover tube 21 at predetermined intervals over the range from the distal end to the proximal end side even if the spiral tube 82 under a rotating condition gets twisted in a winding body-cavity inner tube line. Furthermore, the introductory tube 20B is set so that a distance between the inner peripheral surface of the spiral tube 82 and the outer peripheral surface of the elastic cover tube 21 is not constant over the range from the distal end to the proximal end side.

Accordingly, the introductory tube 20B provides not only the same effect as the introductory tube 20A in the sixth embodiment, but also high resistance to collapse and breakage in the diametrical direction by the plate-shaped metal member 81.

In the present embodiment, the present invention is applied to a configuration of a disposable sheath as the introductory tube 20B, but the present invention is not limited to this. Naturally, the present invention may be applied to a type formed integrally with an endoscope insertion portion as an introductory tube and a tubular-shaped tube formed so as to be harder than a flexible tube portion of an endoscope what is called over-tube. It is sufficient that the friction reduction section reduces a contact resistance between an inner peripheral surface of the spiral tube 82 and a non-rotation portion and provides a significant propulsion function.

The respective embodiments comprise a friction reduction section over the range from the distal end to the proximal end, but the friction reduction section may be provided at a part of the cover tube 21 without being provided over the range from the distal end to the proximal end. Specifically, irregularities may be provided at a part of the cover tube 21.

The present invention is not limited to the embodiments described above, and it is to be understood that various modifications may be made without departing from the spirit or scope of the invention.

### INDUSTRIAL APPLICABILITY

An endoscope insertion portion and an endoscope system according to the present invention are well-suited for introduction of the endoscope insertion portion into a complicated body cavity, by providing a satisfactory propulsion function, by reducing friction between a propulsive force generation portion and an insertion portion.

1. An endoscope insertion portion comprising: an insertion portion capable of being inserted into a subject; a propulsive force generation section mounted on an outer peripheral surface of the insertion portion and rotating around a longitudinal axis of the insertion portion; and a friction reduction section provided between the propulsive force generation section and the outer peripheral surface of the insertion portion and reducing a contact resistance between the outer peripheral surface and the propulsive force generation section.

2. The endoscope insertion portion according to claim 1, wherein the propulsive force generation section is constituted of a spiral-shaped portion.

3. The endoscope insertion portion according to claim 1, wherein the friction reduction section is a ring member provided on an outer peripheral surface of the insertion portion.

4. The endoscope insertion portion according to claim 1, wherein the friction reduction section is a protrusion portion provided on an outer peripheral surface of the insertion portion.

5. The endoscope insertion portion according to claim 1, wherein the friction reduction section is a groove provided on an outer peripheral surface of the insertion portion.

6. The endoscope insertion portion according to claim 1, wherein the friction reduction section is a winding portion wound with metal strand on an outer peripheral surface of the insertion portion.

7. The endoscope insertion portion according to claim 1, wherein the friction reduction section is a covering portion provided on an outer peripheral surface of the insertion portion.

8. The endoscope insertion portion according to claim 1, wherein the friction reduction portion is an inner protrusion portion in which a part of the propulsive force generation portion protruded in an inside-diameter direction is formed between the propulsive force generation portion and an outer peripheral surface of the insertion portion.

9. The endoscope insertion portion according to claim 2, wherein the friction reduction portion is lubricant provided between an inner peripheral surface of the spiral-shaped portion and an outer peripheral surface of the insertion portion.

10. An endoscope system comprising an endoscope insertion portion according to claim 1 and a rotation device for rotating the propulsive force generation section of the endoscope insertion portion around the longitudinal axis.

11. An endoscope system comprising: a slender and flexible endoscope insertion portion; a flexible insertion portion guide member mounted on an outer periphery side of the endoscope insertion portion and formed with a rotatable spiral-shaped portion on an outer peripheral surface; a rotation device for rotating the spiral-shaped portion of the insertion portion guide member around the longitudinal axis; and a friction reduction section for reducing a contact resistance between the spiral-shaped portion rotated by the rotation device and an outer periphery of the insertion portion guide member.

12. The endoscope system according to claim 11, wherein the friction reduction section is a ring member provided on the outer periphery of the insertion portion guide member.

13. The endoscope system according to claim 11, wherein the friction reduction section is a protrusion portion provided on the outer periphery of the insertion portion guide member or a groove portion formed on the outer periphery of the insertion portion guide member.

14. The endoscope system according to claim 11, wherein the friction reduction section is a winding portion wound around the outer periphery of the insertion portion guide member.

15. The endoscope system according to claim 11, wherein the friction reduction section is a covering portion covering the outer periphery of the insertion portion guide member.

16. The endoscope system according to claim 11, wherein the friction reduction portion is lubricant provided between an inner peripheral surface of the spiral-shaped portion and the outer periphery of the insertion portion guide member.

17. The endoscope system according to claim 11, wherein the friction reduction portion is an inner protrusion portion in which a part of the spiral-shaped portion protruded in an inside-diameter direction is formed between the spiral-shaped portion and the outer periphery of the insertion portion guide member.

18. An endoscope insertion portion comprising: an insertion portion capable of being inserted into a subject; a propulsive force generation section fitted onto the insertion portion rotatably around the longitudinal axis of the insertion portion and self-propelling the insertion portion inserted into a body cavity by rotation; and a friction reduction section for reducing a contact resistance between an inner peripheral surface of the propulsive force generation section and an outer peripheral surface of the insertion portion by setting a distance between the inner peripheral surface of the propulsive force generation section and the outer peripheral surface of the insertion portion not being constant.

19. The endoscope insertion portion according to claim 18, wherein the friction reduction section is formed with a plurality of irregularities provided on an inner peripheral surface of the propulsive force generation section or an outer peripheral surface of the insertion portion.

20. The endoscope insertion portion according to claim 19, wherein the friction reduction section is formed with the plurality of irregularities consecutively provided in the longitudinal-axis direction.

21. The endoscope insertion portion according to claim 19, wherein the propulsive force generation section is formed with the plurality of irregularities provided over the whole range from the distal end to the proximal end by rotating the proximal end to transmit a torque of the proximal end portion to the distal end side and rotate with respect to the insertion portion.
